(19) United States
(12) Patent Application Publication
(10) Pub. No.: US 2008/0021074 A1
(43) Pub. Date: Jan. 24, 2008

(54) Pharmaceutical Compositions and Related Methods of Treatment

(75) Inventor: Steve Cartt, Union City, CA (US)

Correspondence Address: WILSON SONSINI GOODRICH & ROSATI, 650 PAGE MILL ROAD, PALO ALTO, CA, 94304-1050, US

(73) Assignee: QUESTCOR PHARMACEUTICALS, INC., Union City, CA (US)

(21) Appl. No.: 11/771,696

(22) Filed: Jun. 29, 2007

Related U.S. Application Data
(60) Provisional application No. 60/806,153, filed on Jun. 29, 2006.

(51) Int. Cl. A61K 31/165; A61K 31/195; A61K 31/433; A61P 25/04; A61P 25/26

(52) U.S. Cl. 514/362; 514/567; 514/618

(57) ABSTRACT

Pharmaceutical compositions comprising at least one alpha2-adrenergic agonist or baclofen and at least one alpha1-adrenergic agonist are disclosed. Pharmaceutical compositions comprising tizanidine and modafinil are disclosed. Methods for reducing somnolence, sleepiness, lethargy, dizziness, drowsiness, somnolence, tiredness, lightheadedness, increased weakness, confusion, unsteadiness, clumsiness, or a combination of the symptoms thereof in a human patient; treating pain; and attenuating muscle spasticity, using pharmaceutical compositions comprising at least one alpha2-adrenergic agonist or baclofen and at least one alpha1-adrenergic agonist are disclosed.

### CROSS-REFERENCE

This application claims the benefit of U.S. provisional application Ser. No. 60/806,153 filed Jun. 29, 2006, which is incorporated by reference in its entirety.

### BACKGROUND OF THE INVENTION

The present invention relates to a novel formulation that is capable of displaying one or more beneficial therapeutic effects. Alpha-adrenergic receptors are specific neuro-receptors located in the peripheral and central nervous systems throughout the human body. These receptors are important switches for controlling many physiological functions and, thus, represent important targets for drug development. As early as 1923 it was shown that the adrenergic agent norepinephrine (noradrenaline) and epinephrine (adrenaline) facilitated neuromuscular transmission (Orbeli L. A., Bull. Inst. Sci. Leshaft 6: 194-97 (1923)). Eventually adrenergic receptors may be divided into five main classes: alpha1, alpha2, beta1, beta2, and beta3.

Many alpha-adrenergic drugs have been developed over the past 40 years. Examples include clonidine, phenoxybenzamine and prazosin (treatment of hypertension), naphazoline (nasal decongestant), and apraclonidine (treating glaucoma). Alpha-adrenergic drugs can be broken down into two distinct classes: agonists (clonidine and naphazoline are agonists), which mimic the receptor activation properties of the endogenous neurotransmitter norepinephrine, and antagonists (phenoxybenzamine and prazosin are antagonists), which act to block the effects of norepinephrine. Many of these drugs are effective but also produce unwanted side effects (for example, clonidine produces dry mouth and sedation in addition to its antihypertensive effects).

Prior to 1977, only one alpha-adrenergic receptor was known to exist. Between 1977 and 1988, it was generally accepted by the scientific community that at least two alpha-adrenergic receptors-alpha1, and alpha2—existed in the central and peripheral nervous systems. Many alpha-adrenergic drugs that were developed before 1992 are not selective for any particular alpha-adrenergic receptor, and many of these drugs produce untoward side effects which may be attributed to their poor alpha-adrenergic receptor selectivity.

Tizanidine has been shown as an alpha2-adrenergic agonist which mediates certain neuropathic pain (Leiphart J. W. et al., J. Neurosurg. 101(4):641-47 (2004)). Known uses of tizanidine also include muscle relaxants for treating spasticity and chronic muscle pain and sleep disturbances (Smith H. S, and Barton A. E., Am. J. Hosp. Palliat. Care 17(1): 50-58 (2000)). The most common adverse effects associated with tizanidine therapy are dry mouth and somnolence/drowsiness. U.S. Pat. No. 6,455,557, herein incorporated by reference, discloses a method for reducing somnolence in a patient receiving tizanidine therapy by administering tizanidine in a form of an immediate release multiparticulate composition at or around the time food is consumed. This method has several important limitations and a better way to counteract adverse effects associated with an alpha2-adrenergic agonist such as tizanidine is needed.

On the other hand, modafinil has been shown to act as an alpha1-adrenergic agonist for providing a wakefulness-promoting action (Duteil J. et al., Eur. J. Pharmacol. 180(1): 49-58 (1990)). Known adverse effects associated with modafinil therapy include headache, nausea, nervousness, rhinitis, diarrhea, back pain, anxiety, insomia, dizziness, and dyspepsia. Accordingly, counteracting adverse effects associated with an alpha1-adrenergic agonist such as modafinil is also needed.

### SUMMARY OF THE INVENTION

This invention relates to pharmaceutical compositions comprising one or more alpha2-adrenergic agonist and one or more alpha 1-adrenergic agonist, for treatment of muscle relaxation in a human patient, alleviation of pain in a human patient, alleviation of somatic pain, neuropathic pain, or visceral pain, alleviation of chronic or acute pain, stimulation of the central nervous system in a human patient, and/or induction of hyperactivity and/or hypermotility in a human patient.

This invention also relates to methods of treatment for reducing somnolence in a patient, reducing pain in a patient, and attenuating muscle spasticity in a patient.

In one aspect are pharmaceutical compositions comprising (a) a therapeutically effective amount of at least one alpha2-adrenergic agonist or a pharmaceutically acceptable derivative, and (b) a therapeutically effective amount of at least one alpha1-adrenergic agonist or a pharmaceutically acceptable derivative.

When combined with at least one alpha1-adrenergic agonist, in some embodiments of the invention, the pharmaceutical composition comprises an alpha2-adrenergic agonist in an amount sufficient to at least partially impart muscle relaxation in a human patient. In some embodiments of the invention, the pharmaceutical composition comprises an alpha2-adrenergic agonist in an amount sufficient to at least partially alleviate pain in a human patient. In some embodiments of the invention, the pharmaceutical composition comprises the alpha2-adrenergic agonist in an amount sufficient to at least partially alleviate somatic pain, neuropathic pain, or visceral pain. In some embodiments of the invention, the pharmaceutical composition comprises the alpha2-adrenergic agonist in an amount sufficient to at least partially alleviate chronic or acute pain.

When combined with at least one alpha1-adrenergic agonist, in some embodiments of the invention, the pharmaceutical composition comprises an alpha2-adrenergic agonist selected from the group consisting of tizanidine, clonidine, apraclonidine, lofexidine, dexmedetomidine, guanfacine, alpha-methyldopa, brimonidine, yohimbine, guanabenz, levarterenol, metaraminol, oxymetazoline, and their respective pharmaceutical acceptable derivatives. In some embodiments of the invention, the pharmaceutical composition comprises an alpha2-adrenergic agonist selected from the group consisting of tizanidine, clonidine, apraclonidine, lofexidine, medetomidine, dexmedetomidine, nivazerol, guanfacine, alpha-methyldopa, apraclonidine, xylazine, alpha-methylnorepinephrine, brimonidine, yohimbine, B-HT920 (6-allyl-2-amino-5,6,7,8-tetrohydro-4H-thiazolo-[4,5-d]-azepine), UK-14304 (5-bromo-6[2-imidazoline-2-yl amino]quinoxaline), and their respective pharmaceutically acceptable derivatives. In some embodiments of the invention, the pharmaceutical composition comprises tizanidine. In some embodiments of the invention, the pharmaceutical composition comprises about 0.5 mg to about 60 mg of tizanidine or a pharmaceutically acceptable derivative.

When combined with at least one alpha2-adrenergic agonist, in some embodiments of the invention, the pharmaceutical composition comprises an alpha1-adrenergic agonist in an amount sufficient to at least partially impart stimulation of the central nervous system in a human patient. In some embodiments of the invention, the pharmaceutical composition comprises the alpha1-adrenergic agonist in an amount sufficient to induce mild to moderate hyperactivity and/or hypermotility in a human patient. In some embodiments of the invention, the pharmaceutical composition comprises an alpha1-adrenergic agonist selected from the group consisting of modafinil, phenylephrine, adrafinil, prazosin, methoxamine, midodrine, levarterenol, metaraminol, and their respective pharmaceutical acceptable derivatives. In some embodiments of the invention, the pharmaceutical composition comprises modafinil. In some embodiments of the invention, the pharmaceutical composition comprises about 50 mg to about 700 mg of modafinil or a pharmaceutically acceptable derivative. In some embodiments of the invention, the pharmaceutical composition comprises from about 170 mg to about 425 mg of modafinil or a pharmaceutical acceptable derivative.

In some embodiments of the invention, the pharmaceutical composition comprises tizanidine and modafinil or their pharmaceutically acceptable derivatives. In some embodiments of the invention, the pharmaceutical composition comprises between about 0.5 mg to about 60 mg of tizanidine or a pharmaceutically acceptable derivative, and between about 50 mg to about 700 mg of modafinil or a pharmaceutically acceptable derivative.

In some embodiments of the invention, the pharmaceutical composition is in a form selected from the group consisting of a powder, tablet, bite-disintegration tablet, chewable tablet, buccal tablet, capsule, caplet, troche, effervescent power, rapid-disintegration tablet, aqueous suspension produced from powder, elixir, and syrup. In some embodiments of the invention, the pharmaceutical composition is in the form of a tablet or capsule. In some embodiments of the invention, the tablet comprises at least one coating of an alpha2-adrenergic agonist and at least one coating of an alpha1-adrenergic agonist. In some embodiments of the invention, the capsule comprises particles comprising at least one alpha2-adrenergic agonist and at least one alpha1-adrenergic agonist. In some embodiments of the invention, the pharmaceutical composition comprises a pharmaceutically acceptable excipient wherein the alpha2-adrenergic agonist and the alpha1-adrenergic agonist are homogeneously admixed together.

In another aspect are pharmaceutical compositions comprising (a) a therapeutically effective amount of tizanidine or a pharmaceutically acceptable derivative, (b) a therapeutically effective amount of modafinil or a pharmaceutical acceptable derivative, and (c) a pharmaceutically acceptable excipient.

When combined with modafinil, in a preferred embodiment of the invention, the pharmaceutical composition comprises between about 0.5 mg to about 60 mg of tizanidine or a pharmaceutically acceptable derivative. In another preferred embodiment of the invention, the pharmaceutical composition comprises between about 2 mg to about 20 mg of tizanidine or a pharmaceutically acceptable derivative. In other embodiments of the invention, the pharmaceutical composition comprises about 0.5 mg, 1.0 mg, 1.5 mg, 2.0 mg, 2.5 mg, 3.0 mg, 3.5 mg, 4.0 mg, 4.5 mg, 5.0 mg, 6.0 mg, 7.0 mg, 8.0 mg, 9.0 mg, 10.0 mg, 11.0 mg, 12.0 mg, 13 mg, 15 mg, 17.5 mg, 20 mg, 22.5 mg, 25 mg, 27.5 mg, 30 mg, 32.5 mg, 35 mg, 37.5 mg, 40 mg, 42.5 mg, 45 mg, 47.5 mg, 50 mg, 52.5 mg, 55 mg, 57.5 mg, or 60 mg of tizanidine or a pharmaceutically acceptable derivative, when combined with modafinil, another alpha1-adrenergic agonist, or their respective pharmaceutically acceptable derivatives. In the most preferred embodiment of the invention, the pharmaceutical composition comprises between about 2.0 mg or about 4.0 mg of tizanidine or a pharmaceutically acceptable derivative.

When combined with tizanidine, in a preferred embodiment of the invention, the pharmaceutical composition comprises between about 50 mg to about 700 mg of modafinil or a pharmaceutical acceptable derivative. In another preferred embodiment of the invention, the pharmaceutical composition comprises between about 100 mg to about 300 mg of modafinil or a pharmaceutical acceptable derivative. In other embodiments of the invention, the pharmaceutical composition comprises about 50 mg, 100 mg, 150 mg, 200 mg, 250 mg, 300 mg, 350 mg, 400 mg, 450 mg, 500 mg, 550 mg, 600 mg, 650 mg, or 700 mg of modafinil or a pharmaceutical acceptable derivative, when combined with tizanidine, another alpha2-adrenergic agonist, or their respective pharmaceutically acceptable derivatives. In the most preferred embodiment of the invention, the pharmaceutical composition comprises about 100 mg or about 200 mg of modafinil or a pharmaceutical acceptable derivative. In some embodiments of the invention, the pharmaceutical composition comprises from about 170 mg to about 425 mg of modafinil or a pharmaceutical acceptable derivative. In some embodiments, the pharmaceutical composition comprises about 170 mg, 345 mg, 420 mg or about 425 mg of modafinil or a pharmaceutical acceptable derivative.

In some embodiments of the invention, the pharmaceutical composition is in a form selected from the group consisting of a powder, tablet, bite-disintegration tablet, chewable tablet, buccal tablet, capsule, caplet, troche, effervescent power, rapid-disintegration tablet, aqueous suspension produced from powder, elixir, and syrup. In some embodiments of the invention, the pharmaceutical composition is in the form of a tablet or capsule. In some embodiments of the invention, the tablet comprises at least one coating of tizanidine or a pharmaceutically acceptable derivative, and at least one coating of modafinil or a pharmaceutically acceptable derivative. In some embodiments of the invention, the capsule comprises particles comprising tizanidine or a pharmaceutically acceptable derivative, and/or modafinil or a pharmaceutically acceptable derivative. In some embodiments of the invention, the tizanidine or a pharmaceutically acceptable derivative thereof and modafinil or a pharmaceutically acceptable derivative thereof are homogeneously admixed in the pharmaceutically acceptable excipient.

In another aspect are pharmaceutical compositions comprising (a) a therapeutically effective amount of baclofen or a pharmaceutically acceptable derivative, and (b) a therapeutically effective amount of at least one alpha1-adrenergic agonist or a pharmaceutically acceptable derivative.

When combined with at least one alpha 1-adrenergic agonist, in some embodiments of the invention, the pharmaceutical composition comprises baclofen in an amount sufficient to at least partially impart muscle relaxation in a human patient. In some embodiments of the invention, the pharmaceutical composition comprises baclofen in an amount sufficient at least partially to alleviate pain in a human patient. In some embodiments of the invention, the pharmaceutical composition comprises baclofen in an amount sufficient to at least partially alleviate somatic pain, neuropathic pain, or visceral pain. In some embodiments of the invention, the pharmaceutical composition comprises baclofen in an amount sufficient to at least partially alleviate chronic or acute pain.

When combined with at least one alpha1-adrenergic agonist, in a preferred embodiment of the invention, the pharmaceutical composition comprises between about 0.5 mg to about 50 mg of baclofen or a pharmaceutically acceptable derivative. In other embodiments of the invention, the pharmaceutical composition comprises about 0.5 mg, 1 mg, 5 mg, 10 mg, 15 mg, 20 mg, 25 mg, 30 mg, 35 mg, 40 mg, 45 mg, or 50 mg of baclofen or a pharmaceutically acceptable derivative. In an even more preferred embodiment of the invention, the pharmaceutical composition comprises about 5 mg, 10 mg, or 15 mg of baclofen or a pharmaceutically acceptable derivative, when combined with at least one alpha 1-adrenergic agonist.

When combined with baclofen, in some embodiments of the invention, the pharmaceutical composition comprises the alpha1-adrenergic agonist in an amount sufficient to at least partially impart stimulation of the central nervous system in a human patient. In some embodiments of the invention, the pharmaceutical composition comprises the alpha1-adrenergic agonist in an amount sufficient to induce mild to moderate hyperactivity and/or hypermotility in a human patient. When combined with baclofen, in some embodiments of the invention, the alpha1-adrenergic agonist is selected from the group consisting of modafinil, phenylephrine, adrafinil, prazosin, methoxamine, midodrine, levarterenol, metaraminol, and their respective pharmaceutical acceptable derivatives. In some embodiments of the invention, the pharmaceutical composition comprises modafinil. In some embodiments of the invention, the pharmaceutical composition comprises between about 50 mg to about 700 mg of modafinil or a pharmaceutically acceptable derivative. In some embodiments of the invention, the pharmaceutical composition comprises from about 170 mg to about 425 mg of modafinil or a pharmaceutical acceptable derivative. In some embodiments, the pharmaceutical composition comprises about 170 mg, 345 mg, 420 mg or about 425 mg of modafinil or a pharmaceutical acceptable derivative.

In some embodiments of the invention, the pharmaceutical composition comprises baclofen and modafinil or their pharmaceutically acceptable derivatives. In some embodiments of the invention, the pharmaceutical composition with baclofen comprises between about 0.5 mg to 50 mg of baclofen or a pharmaceutically acceptable derivative, and between about 50 mg to 700 mg of modafinil or a pharmaceutically acceptable derivative. In some embodiments of the invention, the pharmaceutical composition comprises from about 170 mg to about 425 mg of modafinil or a pharmaceutical acceptable derivative. In some embodiments, the pharmaceutical composition comprises about 170 mg, 345 mg, 420 mg or about 425 mg of modafinil or a pharmaceutical acceptable derivative.

In some embodiments of the invention, the pharmaceutical compositions comprising baclofen and at least one alpha 1-adrenergic agonist is in a form selected from the group consisting of a powder, tablet, bite-disintegration tablet, chewable tablet, buccal tablet, capsule, caplet, troche, effervescent power, rapid-disintegration tablet, aqueous suspension produced from powder, elixir, and syrup. In some embodiments of the invention, the pharmaceutical composition is in the form of a tablet or capsule. In some embodiments of the invention, the tablet comprises at least one coating of baclofen and at least one coating of an alpha1-adrenergic agonist. In some embodiments of the invention, the capsule comprises particles comprising baclofen and/or an alpha1-adrenergic agonist. In some embodiments of the invention, the pharmaceutical composition comprises a pharmaceutically acceptable excipient wherein baclofen and the alpha1-adrenergic agonist are homogeneously admixed together.

In another aspect are methods of reducing somnolence in a patient receiving an alpha2-adrenergic agonist therapy, comprising administering to the patient a composition comprising (a) a therapeutically effective amount of at least one alpha 1-adrenergic agonist or a pharmaceutically acceptable derivative thereof, and (b) a pharmaceutically acceptable excipient.

When combined with at least one alpha2-adrenergic agonist, in some embodiments of the invention, the composition comprises at least one alpha1-adrenergic agonist in an amount sufficient to at least partially impart stimulation of the central nervous system in a human patient. In some embodiments of the invention, the composition comprises the alpha1-adrenergic agonist in an amount sufficient to induce mild to moderate hyperactivity and/or hypermotility in a human patient, or to decrease or prevent sleepiness, lethargy, dizziness, drowsiness, somnolence, tiredness, lightheadedness, increased weakness due to physical or pharmaceutical therapy or administration, confusion, unsteadiness, clumsiness, or a combination of the symptoms thereof in a human patient. In some embodiments of the invention, the alpha1-adrenergic agonist is selected from the group consisting of modafinil, phenylephrine, adrafinil, prazosin, methoxamine, midodrine, levarterenol, metaraminol, and their respective pharmaceutical acceptable derivatives. In some embodiments of the invention, the composition comprises modafinil. When combined with at least one alpha2-adrenergic agonist, in some embodiments of the invention, the composition comprises between about 50 mg to about 700 mg of modafinil or a pharmaceutically acceptable derivative. In some embodiments of the invention, the composition comprises about 100 mg or about 200 mg of modafinil or a pharmaceutically acceptable derivative and is administered once daily. In other embodiments of the invention, the composition is administered twice daily, three times daily, or more frequently.

In some embodiments of the invention, the method comprises a simultaneous administration of an alpha2-adrenergic agonist and the alpha1-adrenergic agonist or their respective pharmaceutically acceptable derivatives. Simultaneous administration may be achieved by a single formulation or different formulations administered at the same time. In some other embodiments of the invention, the method comprises sequential administrations of at least one alpha2-adrenergic agonist and at least one alpha1-adrenergic agonist or their respective pharmaceutically acceptable derivatives. Sequential administration may be achieved by administering the active ingredients, separately, whether in the same or different formulations within about 5 minutes, 10 minutes, 30 minutes, 1 hour, 1.5 hours, 2 hours, 4 hours, 8 hours, or more, between administration of the first and second actives, and include administration of the first formulation of the first active first, followed by administration of the formulation of the second active, and vice versa. In some embodiments of the invention, the composition consists essentially of an alpha2-adrenergic agonist and an alpha1-adrenergic agonist or their respective pharmaceutically acceptable derivatives. In a preferred embodiment of the invention, the composition comprises a synergistic combination of an alpha2-adrenergic agonist and an alpha1-adrenergic agonist or their respective pharmaceutically acceptable derivatives. When combined with at least one alpha1-adrenergic agonist, in some embodiments of the invention, the alpha2-adrenergic agonist is tizanidine. In some embodiments of the invention, the pharmaceutical compositions is in a form selected from the group consisting of powder, tablet, bite-disintegration tablet, chewable tablet, buccal tablet, capsule, caplet, troche, effervescent power, rapid-disintegration tablet, aqueous suspension produced from powder, elixir, and syrup.

In another aspect are methods of treating pain comprising administering to the patient in need thereof (a) a therapeutically effective amount of at least one alpha2-adrenergic agonist or a pharmaceutically acceptable derivative thereof; and (b) a therapeutically effective amount of at least one alpha 1-adrenergic agonist or a pharmaceutically acceptable derivative thereof.

In some embodiments of the invention relating to a method of treating pain, the pain is somatic pain, neuropathic pain, or visceral pain. In some embodiments of the invention, the pain is chronic pain or acute pain. When combined with at least one alpha 1-adrenergic agonist, in some embodiments of the invention, the alpha2-adrenergic agonist is present in an amount sufficient to at least partially impart muscle relaxation in a human patient. In some embodiments of the invention, the alpha2-adrenergic agonist is selected from the group consisting of tizanidine, clonidine, apraclonidine, lofexidine, dexmedetomidine, guanfacine, alpha-methyldopa, brimonidine, yohimbine, guanabenz, levarterenol, metaraminol, oxymetazoline, and their respective pharmaceutical acceptable derivatives. In some embodiments of the invention, the alpha2-adrenergic agonist is selected from the group consisting of tizanidine, clonidine, apraclonidine, lofexidine, medetomidine, dexmedetomidine, mivazerol, guanfacine, alpha-methyldopa, apraclonidine, xylazine, alpha-methylnorepinephrine, brimonidine, yohimbine, B-HT920 (6-allyl-2-amino-5,6,7,8-tetrohydro-4H-thiazolo-[4,5-d]-azepine), UK-14304 (5-bromo-6 [2-imidazoline-2-yl amino] quinoxaline), and their respective pharmaceutically acceptable derivatives.

When combined with at least one alpha2-adrenergic agonist, in some embodiments of the invention, the alpha1-adrenergic agonist is present in an amount sufficient to at least partially impart stimulation of the central nervous system in a human patient. In some embodiments of the invention, the alpha 1-adrenergic agonist is present in an amount sufficient to induce mild to moderate hyperactivity and/or hypermotility in a human patient, or to decrease or prevent sleepiness, lethargy, dizziness, drowsiness, somnolence, tiredness, lightheadedness, increased weakness due to physical or pharmaceutical therapy or administration, confusion, unsteadiness, clumsiness, or a combination of the symptoms thereof in a human patient. In some embodiments of the invention, the alpha1-adrenergic agonist is selected from the group consisting of modafinil, phenylephrine, adrafinil, prazosin, methoxamine, midodrine, levarterenol, metaraminol, and their respective pharmaceutical acceptable derivatives. In some embodiments of the invention, the alpha2-adrenergic agonist is tizanidine and the alpha1-adrenergic agonist is modafinil.

In some embodiments of the invention, the method comprises simultaneous administration of an alpha2-adrenergic agonist and an alpha1-adrenergic agonist or their respective pharmaceutically acceptable derivatives. Simultaneous administration may be achieved by a single formulation or different formulations administered at the same time. In some other embodiments of the invention, the method comprises separate, for example, sequential, administration of an alpha2-adrenergic agonist and an alpha 1-adrenergic agonist or their respective pharmaceutically acceptable derivatives. Sequential administration may be achieved by administering different formulations within about 5 minutes, 10 minutes, 30 minutes, 1 hour, 1.5 hours, 2 hours, 4 hours, 8 hours, or more, between administration of the first and second actives, and include administration of the first formulation of the first active first, followed by administration of the formulation of the second active, and vice versa. In some embodiments of the invention, the method uses a composition consisting essentially of an alpha2-adrenergic agonist and an alpha 1-adrenergic agonist or their respective pharmaceutically acceptable derivatives. In a preferred embodiment of the invention, the composition comprises a synergistic combination of an alpha2-adrenergic agonist and an alpha1-adrenergic agonist or their respective pharmaceutically acceptable derivatives. In some embodiments of the invention, the pharmaceutical composition is in a form selected from the group consisting of a powder, tablet, bite-disintegration tablet, chewable tablet, buccal tablet, capsule, caplet, troche, effervescent power, rapid-disintegration tablet, aqueous suspension produced from powder, elixir, and syrup.

In another aspect are methods for attenuating muscle spasticity comprising administering to the patient a composition comprising (a) a therapeutically effective amount of at least one alpha2-adrenergic agonist or a pharmaceutically acceptable derivative thereof, (b) a therapeutically effective amount of at least one alpha1-adrenergic agonist or a pharmaceutically acceptable derivative thereof; and (c) a pharmaceutically acceptable excipient.

When combined with at least one alpha1-adrenergic agonist, in some embodiments of the invention, an alpha2-adrenergic agonist is present in an amount sufficient to at least partially impart muscle relaxation in a human patient. In some embodiments of the invention, the alpha2-adrenergic agonist is selected from the group consisting of tizanidine, clonidine, apraclonidine, lofexidine, dexmedetomidine, guanfacine, alpha-methyldopa, brimonidine, yohimbine, guanabenz, levarterenol, metaraminol, oxymetazoline, and their respective pharmaceutical acceptable derivatives.

When combined with at least one alpha2-adrenergic agonist, in some embodiments of the invention, an alpha1-adrenergic agonist is present in an amount sufficient to at least partially impart stimulation of the central nervous system in a human patient. In some embodiments of the invention, an alpha1-adrenergic agonist is present in an amount sufficient to induce mild to moderate hyperactivity and/or hypermotility in a human patient, or to decrease or prevent sleepiness, lethargy, dizziness, drowsiness, somnolence, tiredness, lightheadedness, increased weakness due to physical or pharmaceutical therapy or administration, confusion, unsteadiness, clumsiness, or a combination of the symptoms thereof in a human patient. In some embodiments of the invention, the alpha1-adrenergic agonist is selected from the group consisting of modafinil, phenylephrine, adrafinil, prazosin, methoxamine, midodrine, levarterenol, metaraminol, and their respective pharmaceutical acceptable derivatives. In some embodiments of the invention, the alpha2-adrenergic agonist is tizanidine and the alpha1-adrenergic agonist is modafinil.

In some embodiments of the invention, the method comprises simultaneous administration of an alpha2-adrenergic agonist and an alpha1-adrenergic agonist or their respective pharmaceutically acceptable derivatives. Simultaneous administration may be achieved by a single formulation or different formulations administered at the same time. In some other embodiments of the invention, the method comprises separate, for example, sequential administration of an alpha2-adrenergic agonist and an alpha 1-adrenergic agonist or their respective pharmaceutically acceptable derivatives. Sequential administration may be achieved by administering different formulations within about 5 minutes, 10 minutes, 30 minutes, 1 hour, 1.5 hours, 2 hours, 4 hours, 8 hours, or more, between administration of the first and second actives, and include administration of the first formulation of the first active first, followed by administration of the formulation of the second active, and vice versa. In some embodiments of the invention, the method comprises administering a composition consisting essentially of an alpha2-adrenergic agonist and an alpha1-adrenergic agonist or their respective pharmaceutically acceptable derivatives. In a preferred embodiment of the invention, the composition comprises a synergistic combination of an alpha2-adrenergic agonist and an alpha1-adrenergic agonist or their respective pharmaceutically acceptable derivatives. In some embodiments of the invention, the pharmaceutical composition is in a form selected from the group consisting of powder, tablet, bite-disintegration tablet, chewable tablet, buccal tablet, capsule, caplet, troche, effervescent power, rapid-disintegration tablet, aqueous suspension produced from powder, elixir, and syrup.

In some embodiments of the invention, the method described further comprises administering to a patient in need thereof a therapeutically effective amount of baclofen or a pharmaceutically acceptable derivative thereof in addition to administering at least one alpha2-adrenergic agonist and at least one alpha1-adrenergic agonist or their respective pharmaceutically acceptable derivatives.

In some embodiments of the invention, an intrathecal pump system may contain a therapeutically effective amount of at least one alpha2-adrenergic agonist and a therapeutically effective amount of at least one alpha1-adrenergic agonist or their respective pharmaceutically acceptable derivatives. In some embodiments of the invention, an intrathecal pump system may contain a therapeutically effective amount of baclofen, a therapeutically effective amount of at least one alpha1-adrenergic agonist, and optionally a therapeutically effective amount of at least one alpha2-adrenergic agonist, or their respective pharmaceutically acceptable derivatives.

In another aspect are pharmaceutical compositions comprising (a) a therapeutically effective amount of at least one alpha2-adrenergic agonist or a pharmaceutically acceptable derivative thereof; (b) a therapeutically effective amount of at least one alpha 1-adrenergic agonist or a pharmaceutically acceptable derivative thereof, and (c) a therapeutically effective amount of at least one compound metabolized by CYP2D6 or a pharmaceutically acceptable derivative thereof. In some embodiments of the invention, the compound metabolized by CYP2D6 is selected from the group consisting of diazepam, phenyloin and propranolol.

In another aspect are methods of treatment comprising administering to a patient in need thereof (a) a therapeutically effective amount of at least one alpha2-adrenergic agonist or a pharmaceutically acceptable derivative thereof, (b) a therapeutically effective amount of at least one alpha 1-adrenergic agonist or a pharmaceutically acceptable derivative thereof, and (c) a therapeutically effective amount of at least one compound metabolized by CYP2D6 or a pharmaceutically acceptable derivative thereof. In some embodiments of the invention, the compound metabolized by CYP2D6 is selected from the group consisting of diazepam, phenyloin and propranolol.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

While preferred embodiments of the present invention are shown and described herein, it will be apparent to those skilled in the art that such embodiments are provided by way of example only. Numerous variations, changes, and substitutions will now occur to those skilled in the art without departing from the scope of the invention as defined by the appended claims. It should be understood that various alternatives to the embodiments of the invention described herein may be employed in practicing the invention. It is intended that the following claims define the scope of the invention and that methods and compositions within the scope of these claims and their equivalents be covered thereby.

### GLOSSARY OF TERMS

The term “pharmaceutical agent” refers to any agent which imparts or is intended to impart a therapeutic effect and is used or indicated for use as a pharmaceutical. Pharmaceutical agents may be used in the treatment, diagnosis, modulation, or prevention of a diseased state or symptom thereof. One of skill in the art is able to select appropriate pharmaceutical agents when addressing a particular disease or symptom. Exemplary pharmaceutical agents contemplated within the scope of the invention are provided in the following references (the disclosures of all of which are hereby incorporated by reference): Lippincott et al., Remington's Pharmaceutical Sciences: The Science and Practice of Pharmacy, 20th Ed., Williams and Wilkins Publishing, Baltimore (2000); and Lewis et al., Hawley's Condensed Chemical Dictionary, 14th Ed., John Wiley Publishing, New York (2001).

The term “effective amount” refers to any amount sufficient to achieve a desired activity or result. In relation to a pharmaceutical agent, effective amounts can be dosages that are recommended in the modulation of a diseased state or symptom thereof. Effective amounts differ depending on the pharmaceutical agent used and the route of administration employed. Effective amounts are routinely varied or optimized taking into consideration various factors of a particular patient, such as age, weight, gender, etc.

The term “treating” as used herein, refers to reversing, alleviating, inhibiting or slowing the progress of, or preventing the disorder or condition to which such term applies, or one or more symptoms of such disorder or condition. The term “treatment”, as used herein, refers to the act of treating, as “treating” is defined immediately above. Also the terms treating and treatment include at least partial achievement of desired effect or improvement.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The term “therapeutically effective amount” is intended to include an amount of a compound useful in the present invention or an amount of the combination of compounds claimed. The combination of compounds is preferably, but not necessarily, a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general and optionally, a synergistic effect is most clearly demonstrated at suboptimal concentrations of the compounds. Synergy can be in terms of lower cytotoxicity, increased activity, or some other beneficial effect of the combination compared with the individual components. The term “synergistic combination” refers to a synergistic effect of a combination of compounds, for example, for enabling administration at a lower amount of any compound than its full strength amount without the combination for achieving a similar therapeutic effect. Alternatively, “synergistic combination” also refers to a compound's capability to mitigate adverse effects associated with another compound. Accordingly, in some embodiments of the invention, the compound with mitigated adverse effects can be administered at a higher dosage to provide a stronger therapeutic effect at the similar level of adverse effect as compared to a lower dosage without the combination.

The term “excipient” refers to a generally pharmaceutically inactive or inert substance used as a diluent or vehicle for a drug. Different forms of drug administration may require a different excipient and a “pharmaceutically acceptable excipient” includes a “pharmaceutically acceptable carrier.” For example, tablets, troches, pills, capsules, and the like, may contain expicients including a binder such as gum tragacanth, acacia, corn starch or gelatin; a disintegrating agent such as corn starch, potato starch, alginic acid; a lubricant such as magnesium stearate; a sweetening agent such as sucrose, lactose or saccharin; and/or a flavoring agent such as peppermint, oil or wintergreen or cherry flavoring. Capsules may contain additional expicient such as a liquid carrier. Syrups or elixirs may contain expicients including a sweetening agent such as sucrose, a preservative such as methyl and propylparabens, a dye and/or flavoring such as cherry or orange flavor.

The term “pharmaceutically acceptable carrier” refers to one or more compatible solid or liquid filler diluents or encapsulating substances. By “compatible” as used herein is meant that the components of the composition are capable of being comingled without interacting in a manner which would substantially decrease the pharmaceutical efficacy of the total composition under ordinary use situations.

The term “coating” refers to a material that is impermeable and insoluble in the fluid of the environment of use, can form films, and does not adversely affect the drug, animal body, or host. Preferably, the coating is impermeable to water and also impermeable to the selected product, drugs, polymer hydration modulating agents, or to other compounds in the device. This impermeable material is insoluble in body fluids and non-erodible or it can be bioerodible after a predetermined period with bioerosion following the end of the active drug release period. In each instance, it is impermeable to solvent and solute(s) and is suitable for construction of the device.

The term “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. In addition, a single-isomer formulation of a racemic compound is also a “pharmaceutically acceptable derivative” within the scope of the invention. For example, NUVIGIL™ is a single-isomer formulation of the racemic compound modafinil, the active pharmaceutical ingredient contained in PROVIGIL® (modafinil), and NUVIGIL™ is a “pharmaceutically acceptable derivative” of PROVIGIL® within the scope of this invention.

Pharmaceutically acceptable salts include, but are not limited to, amine salts, including but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. The pharmaceutically acceptable salts also include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. The pharmaceutically acceptable salts of the compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred.

Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

The term “alpha2-adrenergic agonists” includes chemical entities, such as compounds, ions, complexes and the like, which are effective to act on or bind to alpha2-adrenergic receptors and provide a therapeutic effect. Alpha2-adrenergic agonists purport the agonists themselves and any and all precursors thereof, metabolites thereof and combinations thereof. Alpha2-adrenergic agonists also include entities capable of producing a net sympatholytic response, resulting in increased accommodation, for example, by binding to presynaptic alpha2-receptors on sympathetic postganglionic nerve endings, or to postsynaptic alpha2-receptors on smooth muscle cells. A sympatholytic response is characterized by the inhibition, diminishment, or prevention of the effects of impulses conveyed by the sympathetic nervous system. Alpha2-adrenergic agonists also include compounds that have neuroprotective activity. For example, 5-bromo-6-(2-imidozolin-2-ylamino) quinoxaline is an alpha2-adrenergic agonist which has a neuroprotective activity through an unknown mechanism.

Similarly, the term “alpha1-adrenergic agonists” includes chemical entities, such as compounds, ions, complexes and the like, which are effective to act on or bind to alpha1-adrenergic receptors and provide a therapeutic effect. Alpha1-adrenergic agonists purport the agonists themselves and any and all precursors thereof, metabolites thereof and combinations thereof.

Without limiting the scope of this invention, it is well known in the art that both systemically and neuraxially administered alpha2-adrenergic agonists, such as clonidine and dexmedetomidine, alleviate pain in humans and in animal models. The alpha2-adrenergic agonists typically produce analgesia by a supraspinal (Guo T Z et al, Anesthesiology 75:252-6 (1991)) as well as by a local spinal action (Eisenach J et al, Anesthesiology 78(2): 277-87 (1993)). Unlike local anesthetics, it is believed that alpha2-adrenergic agonists do not substantially change motor or sensory function, and it is believed that unlike opiates they do not produce respiratory depression (Jarvis D A et al, Anesthesiology 76: 899-905 (1992)) or induce drug-seeking behavior (i.e. addiction). As a result of these features, alpha2-adrenergic agonists are attractive candidates for pain management and are effective for the reduction of post-operative pain (Bonnet F et al, Br J Anaesth 63: 465-9 (1989)) and for pain relief during and after childbirth (Eisenach J C et al, Anesthesiology 71: 640-6 (1989); Filos K S et al, Anesthesiology 77: 267-74 (1992)). The scope of this invention is not limited in the mechanisms described above.

Combination Therapy

Compositions and methods for combination therapy are provided herein. Co-administration of both alpha2-adrenergic agonist and alpha 1-adrenergic agonist allow one adrenergic agonist to counteract adverse effects associated with the other adrenergic agonist. A preferred embodiment of the invention is that the co-administration of an alpha2-adrenergic agonist and an alpha1-adrenergic agonist (as a single formulation, separate formulation administered at the same time, or by sequential administration) mitigates adverse effects associated with either or both adrenergic agonists. Another preferred embodiment of the invention is that the co-administration of an alpha2-adrenergic agonist and an alpha1-adrenergic agonist (as a single formulation, separate formulation administered at the same time, or by sequential administration) enhances the efficacy of the individual compounds through a synergistic mechanism, but also diminishes the likelihood of the adverse and unwanted side effects that these drugs can cause when used alone. Sequential administration may be achieved by administering different formulations within about 5 minutes, 10 minutes, 30 minutes, 1 hour, 1.5 hours, 2 hours, 4 hours, 8 hours, or more, between administration of the first and second actives, and include administration of the first formulation of the first active first, followed by administration of the formulation of the second active, and vice versa.

Disorders and adverse effects associated with certain compounds suitable for treatment using compositions and methods of the invention include, but not limited to, pain, muscle spasticity, narcolepsy, somnolence, sleepiness, lethargy, dizziness, drowsiness, tiredness, lightheadedness, increased weakness due to physical or pharmaceutical therapy or administration, confusion, unsteadiness, clumsiness, other sleep disorders, or a combination of the symptoms thereof. An aspect of the invention also relates to treatments for any combination of above-mentioned disorders or adverse effects associated with certain compounds.

The concentrations or dosage amounts employed in the pharmaceutical compositions may be the minimum concentrations required to achieve the desired clinical effect. However, it is common for a physician to determine the actual dosage that will be most suitable for an individual patient, and this dose will vary with the age, weight, medical condition and response of the particular patient. A physician's adjustments of dosages for a particular patient are within the scope of the invention.

Tizanidine

Tizanidine hydrochloride (C₉H₈ClN₅S—HCl) is a alpha2-adrenergic agonist with antinociceptive and antispastic properties. Its chemical name is 5-chloro-4-(2-imidazolin-2-ylamino)-2,1,3-benzothiodiazole hydrochloride. Synthesis of the compound and its myotonolytic properties are disclosed in U.S. Pat. Nos. 3,843,668 and 4,053,617, which are hereby incorporated by reference in their entireties. Tizanidine's alpha2-adrenergic agonist activity presumably reduces spasticity and/or pain by increasing presynaptic inhibition of motor neurons. The effects of tizanidine are greatest on polysynaptic pathways. The overall effect of these actions is thought to reduce facilitation of spinal motor neurons. The imidazoline chemical structure of tizanidine is related to that of the anti-hypertensive drug clonidine and other alpha2-adrenergic agonists. Tizanidine's capacity to reduce increased muscle tone associated with spasticity enable it to be a short-acting drug for the management of spasticity. Tizanidine potentially exhibits utility in neuropathic pain states (e.g., trigeminal neuralgia) (Fromm G H et al., Pain 53(3): 265-71 (1993)) and in conditions with sympathetic maintained pain (Nabeshima T et al., Neuropharmacol. 26(10): 1453-55 (1987)). It also shows good results in the treatment of patients with low back pain (Berry H and Hutchinson D R, J. Intern Med. Res. 16: 83-91 (1988)), tension-type headaches (Fogelholm R and Murro K, Headache 32: 509-13 (1992), and spasticity related cerebral or spinal injury (Nance P W, Arch Neurol. 54: 731-36 (1997); Nance P W et al., Neurology 44 (suppl 9): S44-52 (1994)). Furthermore, in clinical studies comparing its antispastic effects with diazepam and baclofen, tizanidine proved as effective as other antispastic agents while offering a more favorable tolerability profile, especially in the incidence of debilitating muscle weakness (Bass B et al., Can J Neurol Sci. 15:15019 (1988); Bes A et al., Curr Med Res Opin. 10(10): 709-18 (1988)). Common adverse effects of tizanidine include dry mouth, somnolence (drowsiness), asthenia (weakness, fatigue and/or tiredness), dizziness, hypotention, and bradycardia.

Modafinil

Modafinil (C₁₅H₁₅NO₂S) is a wakefulness-promoting agent for oral administration. Its chemical name is 2-[(diphenylmethyl)sulfinyl]acetamide or 2-(benzhydrylsulfinyl)acetamide, which is known as a racemic-compound. Modafinil has been described as presenting a “neuropsychopharmacological spectrum characterized by the presence of excitation with hyperactivity and of hypermotility” (U.S. Pat. No. 4,177,290; herein incorporated by reference). A single administration of modafinil results in increased locomotor activity in mice and increased nocturnal activity in monkeys (Duteil et al., Eur. J. Pharmacol. 180: 49 (1990)). The neuropsychopharmacological profile of modafinil has been distinguished from that of amphetamines (Saletu et al., Int. J. Clin. Pharm. Res. 9: 183 (1989)). Modafinil is thought to modulate the central postsynaptic alpha 1-adrenergic receptor, without participation of the dopaminergic system (Duteil et al). Modafinil is not indicated for complaints of lack of energy or fatigue, but it appears to be very helpful for some patients. Also modafinil has been successfully tested in humans for treatment of narcolepsy and hypersomnia, a disorder in which patients lack the capacity for meaningful sleep and may require ten or more hours per day (Bastuji et al., Prog. Neuro-Psych. Biol Psych. 12: 695 (1988)).

Modafinil is in a class of medications called central nervous system (CNS) stimulants. The use of modafinil is thought to be effective by changing the amounts of certain natural substances in the area of the brain that controls sleep and wakefulness. Modafinil is known to provide memory-improving, mood-brightening, and vigilance enhancement, but its effect is notably different from amphetamines, methylphenidate or cocaine. Modafinil has alpha 1-adrenergic agonist activity and is less likely to cause jitteriness, anxiety, or excess locomotor activity than traditional stimulants. Known adverse effects associated with Modafinil include headache, nausea, nervousness, rhinitis, diarrhea, back pain, anxiety, insomnia, dizziness, and dyspepsia.

The scope of the invention covers commercial brands of modafinil including PROVIGIL® and NUVIGIL™ marketed by Cephalon Inc. PROVIGIL® (available as 100 mg or 200 mg tablets) is a medication to treat excessive sleepiness caused by certain sleep disorders. These sleep disorders include narcolepsy, obstructive sleep apnea/hypopnea syndrome (OSAHS), and shift work sleep disorder (SWSD). Cephalon Inc. filed a New Drug Application (NDA) with the U.S. Food and Drug Administration seeking approval to market NUVIGIL™ (armodafinil) Tablets to improve wakefulness in patients suffering from excessive sleepiness associated with narcolepsy, shift work sleep disorder (SWSD) and obstructive sleep apnea/hypopnea syndrome (OSA/HS). NUVIGIL™ is a single-isomer formulation of modafinil, the active pharmaceutical ingredient contained in PROVIGIL®, and NUVIGIL™ creates the potential for a truly once-a-day wakefulness-promoting medication. As armodafinil is a single-isomer formulation of modafinil, one of skill in the art will recognize that all disclosures related to modafinil herein necessarily include armodafinil.

In addition, the scope of the invention covers other commercial brands of modafinil such as Sparlon™ (Cephalon Inc.). Sparlon™ has a new formulation of modafinil for the treatment of ADHD and can be available as a once a day pill. Sparlon™ may contain doses of from about 170 mg to about 425 mg of modafinil. In some embodiments of the invention, the pharmaceutical composition comprises from about 170 mg to about 425 mg of modafinil or a pharmaceutical acceptable derivative. In some embodiments of the invention, the pharmaceutical composition comprises about 170 mg, 345 mg, 420 mg or about 425 mg of modafinil or a pharmaceutical acceptable derivative.

Baclofen

Baclofen (C₁₀H₁₂ClNO₂) is a muscle relaxant and antispastic agnet. Its chemical name is 4-amino-3-(4-chlorophenyl)-butanoic acid, and is the p-chlorophenyl derivative of γ-amino-butyric acid (GABA). Administration of baclofen relieves the spasms, cramping, and tightness of muscles caused by medical problems such as multiple sclerosis or certain injuries to the spine. Baclofen has not yet been reported to cure these problems, but it may allow other treatment, such as physical therapy, to be more helpful in improving patient's condition. The precise mechanism by which baclofen produces skeletal muscle relaxation in unknown, but may involve its activity at both pre- and post-synaptic GABA-B receptors, which diminishes neurotransmitter release from presynptic terminals (Bowery N G, Ann Rev Pharmacol Toxicol. 33: 10947 (1993)). This agent can be used orally or via an implantable intrathecal pump. Baclofen is believed to be capable of inhibiting both monosynaptic and polysynaptic reflexes at the spinal level, possibly by hyperpolarization of afferent terminals, although actions at supraspinal sites may also occur and contribute to its clinical effect. Although baclofen is an analog of the putative inhibitory neurotransmitter GABA, there is no conclusive evidence that actions on GABA systems are involved in the production of its clinical effects. Typically baclofen is useful for the alleviation of signs and symptoms of spasticity resulting from multiple sclerosis, particularly for the relief of flexor spasms and concomitant pain, clonus, and muscular rigidity. In addition, it may also be used in patients with spinal cord injury and other spinal cord diseases. Common adverse effects of baclofen include drowsiness or unusual tiredness, dizziness or lightheadedness, increased weakness, confusion, unusual constipation, new or unusual bladder symptoms, trouble sleeping, unusual unsteadiness or clumsiness, and fatigue. A small number of patients also experience fainting, hallucinations, skin rash or itching, or severe mood changes. Overdosage of baclofen may created symptoms of vomiting, muscular hypotonia, drowsiness, sudden onset of blurred or double vision, accommodation disorders, coma, respiratory depression, or seizures. Therefore, generally a minimum effective dosage of baclofen is recommended.

Combination Therapy Including Modafinil and Tizanidine

In in vitro studies using primary human hepatocyte cultures, modafinil was shown to slightly induce CYP1A2, CYP2B6 and CYP3A4 in a concentration-dependent manner (see Schwartz J R L, Expert Opinion on Pharmacotherapy 6(1): 115-129 (2005)). Although induction results based on in vitro experiments are not necessarily predictive of response in vivo, caution needs to be exercised when modafinil is co-administered with drugs that depend on these three enzymes for their clearance. Specifically, lower blood levels of such drugs could result. For example, tizanidine has been shown metabolized by CYP1A2 in vitro (see Granfors M T et al., British Journal of Clinical Pharmacology 57(3): 349-353 (2003)). Accordingly, for co-administrations with tizanidine and modafinil, tizanidine's therapeutically effective amount can be expected to require higher dosages of tizanidine than administrations without co-administrated modafinil.

Combination Therapy with Compounds Metabolized by CYP2D6

One aspect of the invention relates to compositions and methods of treatment comprising administering to a patient (a) a therapeutically effective amount of at least one alpha2-adrenergic agonist or a pharmaceutically acceptable derivative thereof; (b) a therapeutically effective amount of at least one alpha1-adrenergic agonist or a pharmaceutically acceptable derivative thereof; and (c) a therapeutically effective amount of at least one compound metabolized by CYP2D6 or a pharmaceutically acceptable derivative thereof. In some embodiments of the invention, the compound metabolized by CYP2D6 is selected from the group consisting of diazepam, phenyloin and propranolol.

CYP2D6 metabolizes a number of antidepressants, antipsychotics, beta-adrenoceptor blockers, and antiarrhythmic drugs. Because modafinil and modafinil sulfone are reversible inhibitors of the drug-metabolizing enzyme CYP2C19, co-administration of modafinil with drugs such as diazepam, phenyloin and propranolol, which are largely eliminated via that pathway, may increase the circulating levels of those compounds. In addition, in individuals deficient in the enzyme CYP2D6 (i.e., 7-10% of the Caucasian population; similar or lower in other populations), the levels of CYP2D6 substrates such as tricyclic antidepressants and selective serotonin reuptake inhibitors, which have ancillary routes of elimination through CYP2C19, may be increased by co-administration of modafinil.

Diazepam, 7-chloro-1,3-dihydro-1-methyl-5-phenyl-2H-1,4-benzodiazepin-2-one, is indicated for the management of anxiety disorders or for the short-term relief of the symptoms of anxiety. In acute alcohol withdrawal, diazepam may be useful in the symptomatic relief of acute agitation, tremor, impending or acute delirium tremens and hallucinosis. Diazepam is also for the relief of skeletal muscle spasm due to reflex spasm to local pathology such as inflammation of the muscles or joints, spasticity caused by upper motor neuron disorders, athetosis, stiff-man syndrome, or tetanus.

Phenyloin, 5,5-diphenyl-2,4-imidazolidinedione, is an antiepileptic drug which acts on the motor cortex to inhibit spread of seizure activity. Phenyloin also reduces the maximal activity if brain stem centers responsible for the tonic phase of tonic-clonic (grand mal) seizures. Phenyloin is indicated for the control of generalized tonic-clonic (grand mal) and complex partial seizures and prevention and treatment of seizures occurring during or following neurosurgery.

Propranolol HCl, 1-(Isopropylamino)-3-(1-naphthyloxy)-2-propanol hydrochloride, is a beta-adrenergic receptor blocking agent which specifically competes with beta-adrenergic agonist for available receptor sites. Propranolol is indicated in the management of hypertension. It may be used alone or used in combination with other antihypertensive agents, particularly a thiazide diuretic.

Narcolepsy and Somnolence

In accordance with one aspect of the invention, pharmaceutical compositions and methods disclosed herein are useful for reducing somnolence in a patient receiving an alpha2-adrenergic agonist therapy. The method comprises administering to the patient a composition comprising (a) a therapeutically effective amount of at least one alpha1-adrenergic agonist or a pharmaceutically acceptable derivative thereof, and (b) a pharmaceutically acceptable excipient.

In some embodiments of the invention, the composition comprises an alpha1-adrenergic agonist in an amount sufficient to at least partially impart stimulation of the central nervous system in a human patient. In some embodiments of the invention, the composition comprises an alpha1-adrenergic agonist in an amount sufficient to induce mild to moderate hyperactivity and/or hypermotility in a human patient, or to decrease or prevent sleepiness, lethargy, dizziness, drowsiness, somnolence, tiredness, lightheadedness, increased weakness due to physical or pharmaceutical therapy or administration, confusion, unsteadiness, clumsiness, or a combination of the symptoms thereof in a human patient.

In some embodiments of the invention, the alpha1-adrenergic agonist is selected from the group consisting of modafinil, phenylephrine, adrafinil, prazosin, methoxamine, midodrine, levarterenol, metaraminol, and their pharmaceutical acceptable derivatives. Table I lists the indications and dose ranges for these preferred alpha1-adrenergic agonists.

TABLE I
Alpha1-adrenergic agonists

| | | Target | |
|---|---|---|---|
| Drug Name | Indication | Receptor | Dose Range |
| Modafinil | Excessive | Alpha1 | MRTD = 3.33* |
| | sleepiness with | | Oral: (1) Adults - initial dose 200 mg once a day; (2) |
| | narcolepsy, | | Children - dose determined by a doctor |
| | obstructive sleepapnea/ | | |
| | hypopnea | | |
| | syndrome, shift | | |
| | work sleep | | |
| | disorder. | | |
| Phenylephrine | Severe | Alpha1 | MRTD = 0.833* |
| | hypotension and | | Subcutaneous or intramuscular injection: initial dose |
| | shock - including | | not exceed 5 mg, usual dose 1-10 mg. |
| | drug-related | | IV infusion: initial dose not exceed 0.5 mg, usual dose |
| | hypotension, | | 0.1-0.5 mg; Injections should not be repeated more |
| | spinal anesthesia - | | often than every 10 to 15 minutes. |
| | hypotension, | | Nose jelly: (1) Adults - small amount every 3-4 hrs; |
| | paroxysmal | | (2) Children - not recommend |
| | Supraventricular | | Nose drops - (1) Adults and children 12 years or |
| | tachycardia, | | older- 2-3 drops of 0.25 or 0.5% solution every 4 hrs; |
| | nasal congestion, | | (2) Children 6-12 years old - 2-3 drops of 0.25% |
| | dilate eye's pupil | | solution every 4 hrs; (3) Children 2 to 6 years old - 2-3 |
| | | | drops of 0.125 or 0.16% solution every 4 hrs; (4) |
| | | | Children less than 2 years old - dose determined by a |
| | | | doctor |
| | | | Nose spray: (1) Adults and children 12 years or older - |
| | | | 2-3 sparys of 0.25 or 0.5% solution every 4 hrs; (2) |
| | | | Children 6-12 years old - 2-3 drops of 0.25% solution |
| | | | every 4 hrs; (3) Children less than 6 years old - dose |
| | | | determined by a doctor |
| | | | Eye drops: (1) Adults - one drop of 2.5 or 10% solution |
| | | | in the eye from once a day to three times a day; (2) |
| | | | Children - one drop of 2.5% solution in the eye from |
| | | | once a day to three times a day |
| Adrafinil | Stimulant and | Alpha1 | Oral: One or two tablets (300 mg each) are taken twice |
| | anti-depressant\hat{ } | | a day |
| Prazosin | Hypertension | Alpha1 | Oral: (1) Adults - initial dose 0.5 or 1 mg 2-3 times a |
| | | | day, slowly increase to 6-15 mg a day; |
| | | | (2) Children - 50 to 400 μg/kg body weight (0.05 to 0.4 mg) |
| | | | a day |
| Methoxamine | Hypotension, | Alpha1 | MRTD = 0.333* |
| | Supraventricular | | Intramuscular injection or IV infusion: initial dose 3-5 mg |
| | paroxysmal | | then continuous infusion begin at 5 ug/min (dilute |
| | tachycardia | | 40 mg/250 ml); for Supraventricular tachycardia, IV 10 mg |
| Midodrine | Symptomatic | Alpha1 | MRTD = 0.25* |
| | orthostatic | | Oral: (1) Adults - 10 mg 3 times a day every 4 hrs |
| | hypotension | | during daytime; (2) Children - dose determined by a |
| | | | doctor |
| Levarterenol | Cardiac arrest | Alpha1, | IV infusion: (1) Adults - initial dose 8-12 μg/min and |
| | and profound | Alpha2, | titrate to desired response; (2) Children - initial dose |
| | hypotension | Beta1 | 0.05-0.1 μg/kg/min and titrate to desired response. |
| Metaraminol | Severe | Alpha1, | MRTD = 8.33* |
| | hypotension | Alpha2 | Intramuscular or Subcutaneous Injection (for |
| | | | prevention of hypotension): 2 to 10 mg; |
| | | | IV Infusion (for adjunctive treatment of hypotension): |
| | | | 15 to 100 mg in 500 mL of NaCl Injection or 5% |
| | | | Dextrose Injection. |

*Maximum Recommended Therapeutic Dose (MRTD) (mg/kgbw/day)
\hat{ }Adrafinil does not currently have FDA approval in the United States, although it is used in France and elsewhere in Europe.

Somnolence is a major adverse effect associated with alpha2-adrenergic agonists such as tizanidine. Although it is apparent for an ordinary person skilled in the art to identify somnolence and narcolepsy, narcolepsy is generally characterized by intermittent sleep attacks, persistent, excessive daytime sleepiness and abnormal rapid eye movement (“REM”) sleep manifestations, such as sleep-onset REM periods, cataplexy, sleep paralysis and hypnagogic hallucinations (Assoc. of Sleep Disorders Centers, Sleep 2:1 (1979)). Most patients with narcolepsy also have disrupted nocturnal sleep (Montplaisir, in Guilleminault et al. eds., Narcolepsy, Spectrum Pub., New York, pp. 43-56). Pathological somnolence, whether due to narcolepsy or other causes, is disabling and potentially dangerous. Causes of pathological somnolence, other than narcolepsy, include chronic sleep loss (Carskadon et al., Sleep, 5:S73 (1982); Carskadon et al., Psychophysiology, 18:107 (1981)); sleep apnea (Kryger et al., Principles and Practice of Sleep Medicine, W. B. Saunders Co., Philadelphia, Pa. (1989)); and other sleep disorders (International Classification of Sleep Disorders: Diagnostic and Coding Manual, American Sleep Disorder Association, Rochester, Minn. (1990)). Whether due to narcolepsy or other causes, pathological somnolence produces episodes of unintended sleep, reduced attention, and performance errors. Consequently, it is linked to a variety of transportation and industrial accidents (Mitler et al., Sleep 11:100 (1988)). Excessive sleepiness is the primary symptom—and often the most debilitating feature—patients experience with obstructive sleep apnea/hypopnea syndrome (OSA/HS), shift work sleep disorder (SWSD) and narcolepsy. Associated with a reduction of activity in the cerebral cortex of the brain, the defining characteristic of excessive sleepiness is a consistent inability to stay awake and alert enough to safely and successfully accomplish tasks of daily living. Persons experiencing excessive sleepiness who seek medical attention typically complain of fatigue, tiredness, lapses of attention, lack of energy, low motivation, difficulty concentrating, disrupted sleep, snoring or difficulties at work. A therapeutic agent that reduces or eliminates pathological somnolence would have important implications not only for individual patients, but also for public health and safety.

Sleep Disorders

Although adverse effects associated with alpha2-adrenergic agonists including sleep disorders can be identified by an ordinary person skilled in the art, general criteria for sleep disorder have been published by the American Sleep Disorder Association and the American Psychiatric Association. Various tests have been employed to evaluate sleep disorders, including Multiple Sleep Latency Test (MSLT), Maintenance of Wakefulness Test (MWT), Epworth Sleepiness Scale (ESS; a series of questions designed to assess the degree of sleepiness in everyday situations), Steer Clear Performance Test (SCPT; a computer-based evaluation of a patient's ability to avoid hitting obstacles in a simulated driving situation), standard noctunal polysomnography, and use of a patient's daily sleep log.

The MSLT is an objective daytime polysomnographic assessment of a patient's ability to fall asleep in an unstimulating environment, and measures latency (in minutes) to sleep onset averaged over 4 test sessions at 2-hour intervals following nocturnal polysomnography. For each test session, the subject is told to lie quietly and attempt to sleep. Each test session is terminated after 20 minutes if no sleep occurring or 15 minutes after sleep onset. The MWT test measures latency (in minutes) to sleep onset averaged over 4 test sessions at 2 hour intervals following nocturnal polysomnography. For each test session, the subject is asked to attempt to remain awake without using extraordinary measures. Each test session is terminated after 20 minutes if no sleep occurred or 10 minutes after sleep onset. Patients are rated by evaluators who have no access to any data about the patients other than a measure of their baseline severity. Evaluators are not given any specific guidance about the criteria they are to apply when rating patients. A patient's overall disease status can also be measured by the Clinical Global Impression of Change (CGI-C) during drug treatment.

Hyperactivity and Hypermotility

An aspect of the invention is to counteract the somnolence adverse effect associated with alpha2-adrenergic agonists with hyperactivity and hypermotility effects associated with alpha1-adrenergic agonists. One definition of hyperactivity is defined as excessive physical activity or movements that have no purpose and are increased in speed. Hyperactivity can also be described as a state in which a person is abnormally easily excitable and exuberant. Strong emotional reactions and a very short span of attention are also typical for a hyperactive person. Hyperactivity is a state of excessive muscular activity. This term is also used to describe a situation when a particular portion of the body is excessively active, such as when a gland produces too much of its particular hormone. Hyperactive behavior commonly refers to a group of characteristics, including constant activity, easy distractibility, impulsiveness, inability to concentrate, aggressiveness, and similar behaviors. Typical behaviors may include fidgeting or constant moving, wandering, excessive talking, and difficulty participating in quiet activities (such as reading). As can be seen from the above, hyperactivity is not easily defined, because it often depends on the tolerance of the observer. Behavior that seems excessive to one observer may not seem excessive to another. However, certain children—when compared to others—are clearly far more active, which can become a problem if it interferes with school work or making friends. On the other hand, hypermotility is defined as increased, abnormal or excessive movement of all or part of the gastrointestinal tract.

Modafinil has been described as presenting a “neuropsychopharmacological spectrum characterized by the presence of excitation with hyperactivity and of hypermotility; and by the absence of stereotypy (except in strong doses) and of potentialisation of the effects of apomorphine and amphetamine” as disclosed in U.S. Pat. No. 4,177,290, which is hereby incorporated by reference in its entirety. Other methods of measuring or diagnosing stimulation of central nervous system, hyperactivity, and hypermotility are also discussed in Mandryk M. et al., Pharmacological Reports 57: 55-60 (2005) and Danilczuk Z et al., Polish J. of pharmacol. 53: 467-73 (2001). Thus, assays for measuring stimulation of the central nervous system, hyperactivity, and hypermotility can be performed as disclosed in U.S. Pat. No. 4,177,290 and references cited herein.

Pain

In accordance with another aspect of the invention, pharmaceutical compositions and methods disclosed herein are useful for treating pain. Although other assays or methods of measurement will be apparent to those of skill in the art, the method for treating pain disclosed comprises the step of administering to a patient in need thereof

(a) a therapeutically effective amount of at least one alpha2-adrenergic agonist or a pharmaceutically acceptable derivative thereof; and

(b) a therapeutically effective amount of at least one alpha 1-adrenergic agonist or a pharmaceutically acceptable derivative thereof.

When combined with at least one alpha1-adrenergic agonist, in some embodiments of the invention, an alpha2-adrenergic agonist is present in an amount sufficient to at least partially alleviate pain. In some embodiments of the invention, the pain is somatic pain, neuropathic pain, or visceral pain. In some embodiments of the invention, the pain is chronic pain or acute pain. In some embodiments of the invention, an alpha2-adrenergic agonist is present in an amount sufficient to at least partially impart muscle relaxation in a human patient. In some embodiments of the invention, the alpha1-adrenergic agonist is present in an amount sufficient to at least partially impart stimulation of the central nervous system in a human patient. In some embodiments of the invention, the alpha1-adrenergic agonist is present in an amount sufficient to induce mild to moderate hyperactivity and/or hypermotility in a human patient, or to decrease or prevent sleepiness, lethargy, dizziness, drowsiness, somnolence, tiredness, lightheadedness, increased weakness due to physical or pharmaceutical therapy or administration, confusion, unsteadiness, clumsiness, or a combination of the symptoms thereof in a human patient.

When combined with at least one alpha1-adrenergic agonist, in some embodiments of the invention, the alpha2-adrenergic agonist is selected from the group consisting of tizanidine, clonidine, apraclonidine, lofexidine, dexmedetomidine, guanfacine, alpha-methyldopa, brimonidine, yohimbine, guanabenz, levarterenol, metaraminol, oxymetazoline, and their respective pharmaceutical acceptable derivatives. Table II lists the indications and dose ranges for these preferred alpha2-adrenergic agonists.

TABLE II
Alpha2-adrenergic agonists

| | | Target | |
|---|---|---|---|
| Drug Name | Indication | Receptor | Dose Range |
| Tizanidine | Muscle | Alpha2 | MRTD = 0.6* |
| | spasticity | | Oral: (1) Adults - 6-8 mg every 6-8 hrs; maximum |
| | | | dose 36 mg a day; (2) Children - dose determined by a |
| | | | doctor |
| Clonidine | Hypertension | Alpha2 | MRTD = 0.04* |
| | | | Epidural Injection: 30 mcg/hr continuous infusion; |
| | | | dosage rates above 40 mcg/hr is limited |
| | | | Tablet: (1) Adults - initial dose 0.1 mg tablet twice |
| | | | daily; Maintenance dose most commonly employed |
| | | | ranges from 0.2 mg to 0.6 mg per day given in divided |
| | | | doses; |
| | | | (2) Children - 5-10 μg/kg/day in divided doses every |
| | | | 8-12 hrs; increase gradually at 5 - to 7-day intervals to |
| | | | 25 μg/kg/day in divided doses every 6 hours; |
| | | | maximum: 0.9 mg/day |
| Apraclonidine | Glaucoma; | Alpha2 | Eye Drops: (1) Adults - one drop in each eye 2-3 |
| | ocular | | times a day (0.5% apraclonidine for glaucoma) or one |
| | hypertension | | drop in the affected eye one hour before surgery, then |
| | before and | | one drop in the same eye after surgery (1% |
| | after eye | | apraclonidine for eye surgery); |
| | surgery | | (2) Children dose determined by a doctor |
| Lofexidine | Opiate | Alpha2 | MRTD = 0.04* |
| | detoxification | | Oral: Initial dose 2 tablets (0.4 mg) per day; gradually |
| | | | increased up to a maximum of 12 tablets (2.4 mg) per |
| | | | day; gradually decreased as symptom becomes less |
| | | | severe; normally last 7-10 days |
| Dexmedetomidine | Sedation of | Alpha2 | Infusion: initial loading infusion of 1 μg/kg over 10 |
| | patients in | | minutes; maintenance infusion of 0.2-0.7 μg/kg/hr; |
| | intensive care | | continuous infusion not exceed 24 hrs |
| Guanfacine | Hypertension; | Alpha2 | Oral: (1) Adults - Adults - initial dose 1 mg once a |
| | Sleep | | day; may gradually increase dose up to 3 mg a day; (2) |
| | disorders | | Children - dose determined by a doctor |
| Alpha-methyldopa\hat{ } | Hypertension | Alpha2 | MRTD = 33.3* |
| | | | Oral: (1) Adults - Initial dose 250 mg 2-3 times a day; |
| | | | maintenance dose 1-1.5 g/day in 2-4 divided doses; |
| | | | maximum dose 3 g/day; |
| | | | (2) Children - Initial dose 10 mg/kg/day in 2-4 |
| | | | divided doses; maintenance dose up to 65 mg/kg/day; |
| | | | maximum dose 3 g/day. |
| | | | IV: (1) Adults - 250-500 mg every 6-8 hrs; maximum |
| | | | dose: 1 g every 6 hrs; |
| | | | (2) Children - 5-10 mg/kg/dose every 6-8 hrs up to a |
| | | | total dose of 65 mg/kg/24 hrs or 3 g/24 hrs |
| Brimonidine | Glaucoma, | Alpha2 | Eye Drops (0.15% solution): (1) Adults and children 2 |
| | Ocular | | years and older - one drop in the affected eye(s) 3 |
| | hypertension | | times a day about 8 hrs apart; |
| | | | (2) Children less than 2 years - dose determined by a |
| | | | doctor |
| Yohimbine | increase | Alpha2 | Oral: Adult males: one tablet (5.4 mg) 3 times a day |
| | peripheral | | |
| | blood flow, | | |
| | dilate eye's | | |
| | pupil, | | |
| | impotence | | |
| Guanabenz | Hypertension | Alpha2 | Oral: (1) Adults - initial dose 4 mg tablet 2 times a |
| | | | day, maximum dose 32 mg twice a day; |
| | | | (2) children - dose determined by a doctor |
| Levarterenol | Cardiac arrest | Alpha1, | IV infusion: (1) Adults - initial dose 8-12 μg/min and |
| | and profound | Alpha2, | titrate to desired response; (2) Children - initial dose |
| | hypotension | Beta1 | 0.05-0.1 μg/kg/min and titrate to desired response. |
| Metaraminol | Severe | Alpha1, | MRTD = 8.33* |
| | hypotension | Alpha2 | Intramuscular or Subcutaneous Injection (for |
| | | | prevention of hypotension): 2 to 10 mg; |
| | | | IV Infusion (for adjunctive treatment of hypotension): |
| | | | 15 to 100 mg in 500 mL of NaCl Injection or 5% |
| | | | Dextrose Injection. |
| Oxymetazoline | nasal | Alpha2 | Nose drops or spray (0.05% solution): (1) Adults and |
| | congestion, | | children 6 years of age and older - 2 or 3 drops or |
| | minor eye | | sprays every 10-12 hrs; (2) Children up to 6 years of |
| | irritations | | age - dose determined by a doctor; |
| | | | Eye drops: Adults and children 6 years of age and |
| | | | older - 1 drop in the eye every 6 hrs; (2) Children up |
| | | | to 6 years of age - dose determined by a doctor |

*Maximum Recommended Therapeutic Dose (MRTD) (mg/kgbw/day)
\hat{ }Methyldopa's active metabolite is alpha-methylnorepinephrine.

Pain is transmitted though the nervous system and is often associated with a variety of different underlying illnesses or injuries. Pain may be either acute or chronic. Chronic or intractable pain is often endured over many years or decades. Patients suffering from chronic pain often develop emotional problems which can lead to depression and in the worst case, attempted suicide. Long lasting pain often occurs particularly in joints, in muscles, connective tissue and in the back. A patient is considered to have chronic pain when complaints thereof last longer than six months. In the course of time, chronic pain may form an independent clinical syndrome.

Examples of pain conditions that may be treated as contemplated by the invention include, but are not limited to, headaches (e.g., trigerninal neuralgia, sinusitis, cluster headaches, migraines, etc.), low back pain, cancer pain, arthritis pain, muscle spasm pain (for example, muscle cramps), bone pain, pain resulting from burns, pain associated with bumps, pain associated with bruises, inflammatory pain (from an infection or arthritic disorder), pain from obstructions, myofascial pain, pain from nerve trauma (dystrophy/causalgia), phantom limb pain, entrapment neuropathy (e.g., carpal tunnel syndrome), peripheral neuropathy, and pain from wounds, e.g., surgical, accidental, or self-inflicted wounds. The pathophysiology of pain can be broadly divided into three categories: (i) nociceptive pain, (ii) neuropathic pain, and (iii) idiopathic pain. (Willis, W. D., The Pain System. The Neural Basis of Nociceptive Transmission in the Mammalian Nervous System. Pain and Headache, vol. 8, Gildenberg P L (Ed.) Karger Publishers, New York (1985)). Each of these pains can be treated according to compositions and methods of the invention.

Nociceptive pain is the result of receptor stimulation by tissue injury. It involves the normal activation of the nociceptive system by noxious stimuli. Examples of nociceptive pain include sprains, bone fractures, burns, bumps, bruises, inflammation (from an infection or arthritic disorder), obstructions, myofascial pain (which may indicate abnormal muscle stresses) headaches, low back pain, cancer pain, and arthritis pain. In some embodiments of the invention, the compositions or methods disclosed herein are used to prevent or treat nociceptive pain. According to an aspect of the invention, combination therapy using at least one alpha2-adrenergic agonist and at least one alpha1-adrenergic agonist, in a single formulation or sequential administration, can be employed to treat nociceptive pain. Sequential administration may be achieved by administering different formulations within about 5 minutes, 10 minutes, 30 minutes, 1 hour, 1.5 hours, 2 hours, 4 hours, 8 hours, or more, between administration of the first and second actives, and include administration of the first formulation of the first active first, followed by administration of the formulation of the second active, and vice versa.

The second category of pain, neuropathic pain, is the result of an injury or malfunction in the peripheral or central nervous system. Examples of neuropathic pain include post herpetic (or post-shingles) neuralgia, reflex sympathetic dystrophy/causalgia (nerve trauma), components of cancer pain, phantom limb pain, entrapment neuropathy (e.g., carpal tunnel syndrome), and peripheral neuropathy most commonly caused by diabetes or chronic alcohol use. Neuropathic pain is often triggered by an injury, but this injury may or may not involve actual damage to the nervous system. For example, nerves can be infiltrated or compressed by tumors, strangulated by scar tissue, or inflamed by infection, which may cause neuropathic pain. Neuropathic pain may persist for months or years beyond the apparent healing of any damaged tissues. Therefore, neuropathic pain is frequently chronic, not fully reversible, and tends to have a less robust response to treatment with opioids, but may respond to drugs such as anticonvulsants (carbamazepine and valproic acid, and gabapentin) and neuromodulating drugs (including tricyclic antidepressants, such as amitriptyline, imipramine, and desipramine). In some embodiments of the invention, the compositions or methods herein are used to prevent or treat neuropathic pain. According to an aspect of the invention, combination therapy using at least one alpha2-adrenergic agonist and at least one alpha1-adrenergic agonist, in a single formulation or sequential administration, can be employed to treat neuropathic pain. Sequential administration may be achieved by administering different formulations within about 5 minutes, 10 minutes, 30 minutes, 1 hour, 1.5 hours, 2 hours, 4 hours, 8 hours, or more, between administration of the first and second actives, and include administration of the first formulation of the first active first, followed by administration of the formulation of the second active, and vice versa.

The third category of pain, idiopathic pain, is a diagnosis of exclusion in which a patient suffers pain for longer than 6 months for which there is no physical cause and no specific mental disorder. Examples of idiopathic pain include, but are not limited to, arthritis, fibromyalgia, chronic fatigue syndrome, irritable bowel syndrome, interstitial cystitis, vulvadynia, carpal tunnel syndrome, etc. In some embodiments of the invention, the compositions or methods disclosed herein are used to prevent or treat idiopathic pain. According to an aspect of the invention, combination therapy using at least one alpha2-adrenergic agonist and at least one alpha1-adrenergic agonist, in a single formulation or sequential administration, can be employed to treat idiopathic pain. Sequential administration may be achieved by administering different formulations within about 5 minutes, 10 minutes, 30 minutes, 1 hour, 1.5 hours, 2 hours, 4 hours, 8 hours, or more, between administration of the first and second actives, and include administration of the first formulation of the first active first, followed by administration of the formulation of the second active, and vice versa.

For the prevention or treatment of pain, the appropriate dosage of an anti-pain medicament will depend on the type of condition to be treated, as defined above, the severity and course of the disease, whether the agent is administered for preventive or therapeutic purposes or, as a combination with other drugs, previous therapy, the patient's clinical history and response to the agent, and the discretion of the attending physician. The agent is suitably administered to the patient at one time or over a series of treatments. A scaled score of 1 to 10 is used to evaluate treatment efficacy where a score of 10 represents a patient with sever pain discomfort and complete inability to walk. A scaled score of 1 represents a patient experiencing no pain and able to freely walk or move.

Visceral Pain

A common form of pain is visceral pain syndrome, which includes irritable bowel syndrome (IBS), noncardiac chest pain (NCCP), functional dyspepsia, interstitial cystitis, essential vulvodynia, and urethral syndrome. The phrase “visceral pain” refers to pain caused by an abnormal condition of the viscera. It is characteristically severe, crampy, diffuse, and difficult to localize. See Mosby's Medical, Nursing & Allied Health Dictionary, 5th ed. (1998). Visceral pain can include pain in tissue and/or organs located in the viscera as well as pain referred from visceral tissue and/or organs to somatic structures. A common feature of the visceral pain is pain or discomfort arising from the organs and tissues of the thorax, abdomen, and pelvis. Visceral pain is widely believed to be the result of visceral hypersensitivity. One common form of visceral hypersensitivity is visceral hyperalgesia, i.e., increased sensitivity in visceral organs and/or tissues to painful stimuli. See Giamberardino, European Journal of Pain, 3: 77-92 (1999) for a description of the different forms of visceral hyperalgesia. Hyperalgesia is believed to be caused by the “sensitization” of the nervous system. Such sensitization can be a result of changes occurring peripherally (i.e., due to inflammation locally within the skin, muscle, bladder, or in the organs of the gastrointestinal tract), centrally (at the level of the spinal cord, brainstem, thalamus, or cortex), or at both locations. Moreover, acute peripheral sensitization can ultimately lead to a state of chronic central sensitization. The mechanisms underlying central sensitizations are complex and can involve alterations in wide variety of neurotransmitter systems, and not completely understood. Visceral pain may be characterized by pain in the urogenital and rectal area includes vulvodynia, orichialgia, urethral syndrome, penile pain, prostatodynia, coccygodynia, perineal pain, and rectal pain. Several references in the art provide details regarding other abdominal, urogenital, and rectal visceral pain, including diagnostic criteria. See, e.g., Wesselmann et al., Pain, 73:269-294 (1997). The art provides various means for diagnosing the different forms or types of visceral pain. It will be apparent to one of skill in the art that, in addition to the diagnostic criteria described herein, different diagnostic criteria described in other scientific literature may also be used. According to an aspect of the invention, combination therapy using at least one alpha2-adrenergic agonist and at least one alpha 1-adrenergic agonist, in a single formulation or sequential administration, can be employed to treat visceral pain. Sequential administration may be achieved by administering different formulations within about 5 minutes, 10 minutes, 30 minutes, 1 hour, 1.5 hours, 2 hours, 4 hours, 8 hours, or more, between administration of the first and second actives, and include administration of the first formulation of the first active first, followed by administration of the formulation of the second active, and vice versa.

Muscle Relaxation

In accordance with another aspect of the invention, pharmaceutical compositions and methods disclosed herein are useful for attenuating muscle spasticity. The method comprises the step of administering to a patient in need thereof (a) a therapeutically effective amount of at least one alpha2-adrenergic agonist or a pharmaceutically acceptable derivative thereof, and (b) a therapeutically effective amount of at least one alpha1-adrenergic agonist or a pharmaceutically acceptable derivative thereof.

When combined with baclofen, at least one alpha2-adrengeric agonist, or optionally both, in some embodiments of the invention, the alpha1-adrenergic agonist is present in an amount sufficient to at least partially impart stimulation of the central nervous system in a human patient. In some embodiments of the invention, the alpha1-adrenergic agonist is present in an amount sufficient to induce mild to moderate hyperactivity and/or hypermotility in a human patient, or to decrease or prevent sleepiness, lethargy, dizziness, drowsiness, somnolence, tiredness, lightheadedness, increased weakness due to physical or pharmaceutical therapy or administration, confusion, unsteadiness, clumsiness, or a combination of the symptoms thereof in a human patient.

Spasticity is a motor disorder characterized by tight or stiff muscles that might interfere with voluntary muscle movements. Spasticity is believed to be caused by an imbalance of excitatory and inhibitory input in the spinal cord. This imbalance causes hyperactive muscle stretch reflexes. These reflexes result in involuntary spasms and increased muscle tone. The spinal cord is a reflex system, and the most obvious example of reflex is the withdrawal to heat. When movements are brisk and without adequate control from the brain, these movements become rough and uncontrolled reflexes. If the reflexes spread through the body, spasms or arching results. Often these spasms can be painful and/or interrupt sleep.

Muscle relaxation can be measured both objectively (reduction in muscle tone and frequency of spasms in patients using the Ashworth scale and/or Pendulum test), and subjectively (patient and physician assessment by physical examination). With the Ashworth scale, muscle tone is rated on a 5 point scale, with a score of 0 used to describe normal muscle tone. A score of 1 indicates a slight spastic catch while a score of 2 indicates more marked muscle resistance. A score of 3 is used to describe considerable increase in tone, making passive movement difficult. A muscle immobilized by spasticity is given a score of 4. For the Pendulum test, a supine patient is positioned with legs hanging freely over the end of an examination table. The assessor lifts the more spastic leg and then allows it to hang freely at the knee. Three trials of leg movement are videotaped and analyzed in random order by blinded raters. From the digitized movements of the leg, the arc of the first knee swing (in degrees) is measured for statistical analysis. In addition, frequency of spasms provides a good measurement for a drug's effect on muscle relaxation. Assessment of a drug's effect on muscle relaxation is usually carried out at 1, 2, 3, and 6 hours after administration of a short-acting drug such as tizanidine or another suitable alpha2-adrenergic agonist. Occasionally, combination therapy using multiple antispasticity agents is used to avoid exceeding maximum does of either agent. For example, tizanidine and baclofen are shown compatible to be combined without significant pharmacokinetic interaction (Shellenberger M K et al., Drug Metab Disp 27: 201 (1999)). In some embodiments of the invention, the method comprises administering to a patient in need thereof a therapeutically effective amount of baclofen or a pharmaceutically acceptable derivative thereof in addition to administering at least one alpha2-adrenergic agonist and at least one alpha1-adrenergic agonist or their respective pharmaceutically acceptable derivatives.

Intrathecal Pump System

Intrathecal space containscerebrospinal fluid, the fluid surrounding the spinal cord and nerve roots. Drugs like baclofen are often delivered though oral administration, but only a small portion of these drugs reach the spinal fluid where they exert their therapeutic effects. An intrathecal delivery system, such as an intrathecal pump, is an effective way to deliver medicine to the intrathecal space and avoids overdosage adverse effects due to systematic exposure. An intrathecal pump system comprises a catheter and a pump, which is a round metal disc typically about one inch thick and three inches in diameter, and can be surgically placed under the skin of the abdomen near the patient's waistline. The pump stores and releases prescribed amounts of medicine through the catheter. The pump is refilled by inserting a needle through the skin into a filling port in the center of the pump. With a programmable pump, a tiny motor moves the medicine from the pump reservoir through the catheter. Patients must return to their physicians for pump refills and medicine adjustments, typically every two to three months. The pump is taken out and replaced at the end of the battery's life span, which is usually five to seven years. Although an intrathecal pump system is useful to avoid overdosage adverse effect due to systematic exposure, an intrathecal pump system can be employed for combination therapies of the invention. In some embodiments of the invention, an intrathecal pump system may contain a therapeutically effective amount of at least one alpha2-adrenergic agonist and a therapeutically effective amount of at least one alpha 1-adrenergic agonist or their respective pharmaceutically acceptable derivatives. In some embodiments of the invention, an intrathecal pump system may contain a therapeutically effective amount of baclofen, a therapeutically effective amount of at least one alpha1-adrenergic agonist, and optionally a therapeutically effective amount of at least one alpha2-adrenergic agonist, or their respective pharmaceutically acceptable derivatives.

### EXAMPLES

The following prophetic examples are introduced in order that the invention may be more readily understood. They are intended to illustrate the invention but not limit its scope.

### Example 1

### Combination of an Alpha2-Adrenereic Agonist and an Alpha1-Adrenergic Agonist

Zanaflex® tablets (each containing 4 mg tizanidine) are crushed and ground, in a mortar using a pestle, into small particles suitable to be enclosed in capsules. PROVIGIL® tablets (each containing 200 mg modafinil) are crushed and ground, in a mortar using a pestle, into small particles suitable to be enclosed in gelatin capsules (Capsuline®, Capsuline, Inc. Pompano Beach, Fla.). Each capsule is made to contain about 4 mg tizanidine and 200 mg modafinil from the mortars' contents. The capsules are then administered to a patient in need according to the methods disclosed herein. The capsules (as with any formulation described herein) can be administered once daily, twice daily, three times daily, or more frequently.

Alternatively, Zanaflex® capsules (each containing 4 mg tizanidine) are opened and the capsules' contents are mixed with crushed PROVIGIL® tablets in a mortar. Each capsule is made to contain about 4 mg tizanidine and 200 mg modafinil from the mortar's content. The capsules are then administered to a patient in need according to the methods disclosed herein. The capsules (as with any formulation described herein) can be administered once daily, twice daily, three times daily, or more frequently.

### Example 2

### Combination of Beclofen, an Alpha2-Adrenergic Agonist, and an Alpha1-Adrenergic Agonist

Zanaflex® tablets (each containing 4 mg tizanidine) are crushed and ground, in a mortar using a pestle, into small particles suitable to be enclosed in capsules. PROVIGIL® tablets (each containing 200 mg modafinil) are crushed and ground, in a mortar using a pestle, into small particles suitable to be enclosed in capsules. Generic baclofen tablets (each containing 10 mg baclofen) are crushed and ground, in a mortar using a pestle, into small particles suitable to be enclosed in capsules. Each capsule is made to contain about 4 mg tizanidine, 200 mg modafinil, and 10 mg baclofen from the mortars' contents. The capsules are then administered to a patient in need according to the methods disclosed herein. The capsules (as with any formulation described herein) can be administered once daily, twice daily, three times daily, or more frequently.

Alternatively, Zanaflex® capsules (each containing 4 mg tizanidine) are opened and capsules' contents are mixed with crushed PROVIGIL® tablets and crushed generic baclofen tablets in a mortar. Each capsule is made to contain about 4 mg tizanidine, 200 mg modafinil, and 10 mg baclofen from the mortar's content. The capsules are then administered to a patient in need according to the methods disclosed herein. The capsules (as with any formulation described herein) can be administered once daily, twice daily, three times daily, or more frequently.

### Example 3

### Combination of an Alpha2-Adrenergic Agonist and an Alpha1-Adrenergic Agonist

Tenex® tablets (each containing 1 mg guanfacine) are crushed and ground, in a mortar using a pestle, into small particles suitable to be enclosed in capsules. PROVIGIL® tablets (each containing 200 mg modafinil) are crushed and ground, in a mortar using a pestle, into small particles suitable to be enclosed in capsules. Each capsule is made to contain about 1 mg guanfacine and 200 mg modafinil from the mortars' contents. The capsules are then administered to a patient in need according to the methods disclosed herein. The capsules (as with any formulation described herein) can be administered once daily, twice daily, three times daily, or more frequently.

### Example 4

### Combination of More Than One Alpha2-Adrenergic Agonists and More Than One Alpha1-Adrenergic Agonists

Zanaflex® tablets (each containing 4 mg tizanidine) are crushed and ground, in a mortar using a pestle, into small particles suitable to be enclosed in capsules. Tenex® tablets (each containing 1 mg guanfacine) are crushed and ground, in a mortar using a pestle, into small particles suitable to be enclosed in capsules. ProAmatine® tablets (each containing 5 mg midodrine) are crushed and ground, in a mortar using a pestle, into small particles suitable to be enclosed in capsules. PROVIGIL® tablets (each containing 200 mg modafinil) are crushed and ground, in a mortar using a pestle, into small particles suitable to be enclosed in capsules. Each capsule is made to contain about 4 mg tizanidine, 1 mg guanfacine, 5 mg midodrine, and 200 mg modafinil from the mortars' contents. The capsules are then administered to a patient in need according to the methods disclosed herein. The capsules (as with any formulation described herein) can be administered once daily, twice daily, three times daily, or more frequently.

Alternatively, Zanaflex® capsules (each containing 4 mg tizanidine) are opened and capsules' contents are mixed with crushed Tenex® tablets, crushed ProAmatine® tablets, and crushed PROVIGIL® tablets in a mortar. Each capsule is made to contain about 4 mg tizanidine, 1 mg guanfacine, 5 mg midodrine, and 200 mg modafinil from the mortar's content. The capsules are then administered to a patient in need according to the methods disclosed herein. The capsules (as with any formulation described herein) can be administered once daily, twice daily, three times daily, or more frequently.

The following examples show various formulations which can be used for the pharmaceutical compositions disclosed herein. The “active ingredients” in the following examples may comprise at least one alpha2-adrenergic agonist combined with at least one alpha1-adrenergic agonist; baclofen combined with at least one alpha1-adrenergic agonist, optionally also combined with at least one alpha2-adrenergic agonist; at least one alpha1-adrenergic agonist combined with at least one compound metabolized by CYP2D6, optionally also combined with at least one alpha2-adrenergic agonist; or their respective pharmaceutically acceptable derivatives.

### Example 5

A sustained-release dosage form of the active ingredients can be prepared by having the active ingredients surrounded by an interior and an exterior wall, with an exit that allows for administration of the active ingredients to a patient, as described in U.S. Pat. No. 6,245,357; wherein the active ingredients as described herein are substituted for the active ingredient described therein. The sustained-release dosage form can include the active ingredients, and a pharmaceutically acceptable polyethylene oxide carrier, which is coated with a wall comprising ethylcellulose and hydroxypropylcellulose. More specifically, the sustained-release dosage form can also include the active ingredients and a pharmaceutically acceptable polyethylene oxide carrier, which is coated with an interior wall comprising ethyl cellulose and hydroxypropylcellulose, and an exterior wall containing cellulose acetate. The sustained-release dosage form can also be prepared as a dosage form for delivering the active ingredients at a sustained-release rate to a gastrointestinal-lipid-fluid environment. The dosage form includes a composition containing doses of the active ingredients, and a coat that envelopes the composition containing the active ingredients. The coat includes a passage-former that leaves the coat in the presence of fluid, and a wall that surrounds the coat and prevents lipid in the gastrointestinal tract from entering the dosage form. Additional substances that can be included in the above sustained-release dosage forms, as well as methods to make the sustained-release dosage forms, are described in U.S. Pat. No. 6,245,357.

### Example 6

A tablet for controlled release for the active ingredients can be prepared as described in U.S. Pat. No. 6,033,685; wherein the active ingredients as described herein are substituted for the active ingredient described therein. The tablet includes a matrix layer having the active ingredients embedded in a non-swelling, non-gelling hydrophobic matrix; a first barrier layer laminated to a single face of the matrix layer; and an optional second barrier layer laminated to the opposite face of the matrix layer and oppositely disposed to the first barrier layer. The matrix contains up to about 80% of the active ingredients, and from about 5% to about 80% by weight of nonswellable waxes or polymeric material insoluble in aqueous medium. The first and second barrier layers independently include polymeric material exhibiting a high degree of swelling and gelling in aqueous medium, or nonswellable wax or polymeric material insoluble in aqueous medium. Additional substances that can be included in the above controlled release tablets, as well as methods to make the controlled release tablets, are described in U.S. Pat. No. 6,033,685.

### Example 7

A pharmaceutical composition for extended release of the active ingredients in a gastrointestinal environment can be prepared as described in U.S. Pat. No. 6,010,718; wherein the active ingredients as described herein are substituted for the active ingredient described therein. The composition includes the active ingredients and a pharmaceutically acceptable polymer so that, when ingested orally, the composition induces statistically significantly lower Cmax in the plasma than an immediate release composition of the active ingredients. The pharmaceutical composition maintains bioavailability and minimum concentration substantially equivalent to that of an immediate release composition of the active ingredient achieved by multiple dosing. Additional substances that can be included in the above extended release pharmaceutical compositions, as well as methods to make the extended release pharmaceutical compositions, are described in U.S. Pat. No. 6,010,718.

### Example 8

Orally administrable pharmaceutical preparations having controlled release of the active ingredients can be prepared as described in U.S. Pat. No. 5,900,425; wherein the active ingredients as described herein are substituted for the active ingredient described therein. Such controlled release pharmaceutical preparations can include the active ingredients in amorphous form as a coprecipitate in a polyvinylpyrrolidone homo or copolymer having a weight average molecular weight of about 15,000 to 1,000,000 and, a release-delaying component containing a gel-forming polymer having a viscosity of at least 15 mPas when measured at a 2% concentration at 20° C. Additional substances that can be included in the orally administrable extended release pharmaceutical compositions, as well as methods to make the orally administrable extended release pharmaceutical compositions are described in U.S. Pat. No. 5,900,425.

### Example 9

A tablet form for controlled release of the active ingredients in a dispersion can be prepared as described in U.S. Pat. No. 5,882,682; wherein the active ingredients as described herein are substituted for the active ingredient described therein. The tablet has a compressed core which contains the active agent, a polymer which forms gelatinous microscopic particles upon hydration, and if desired, an agent to modulate the hydration; and a water insoluble coating which adheres to and surrounds the core and contains apertures which provide an area for the hydration and release of the dispersion. The release rate of the active ingredients is a function of the number and size of the apertures in the coating of the tablet. The active ingredients may be prepared for controlled release from a tablet as a dispersion by preparing a compressed core from an admixture containing therapeutically effective amounts of the active ingredients, a polymer which upon hydration forms gelatinous microscopic particles, and a water insoluble, water impermeable polymeric coating. The water insoluble, water impermeable polymeric coating can contain a polymer and a plasticizer, which surrounds and adheres to the core. The polymer can include, e.g., cellulose acetate, cellulose acetate butyrate, ethylcellulose, polyvinylacetate, polyvinyl chloride, polymers of acrylic, methacrylic acid esters, or a combination thereof. The plasticizer can include, e.g., dibutylsebacate, diethylphthalate, triethylcitrate, polyethylene glycol, or a combination thereof. The polymer which upon hydration forms gelatinous microscopic particles can include, e.g., sodium polyacrylate, carboxypolymethylenes, the pharmaceutically acceptable salts thereof, or a combination thereof. The carboxypolymethylenes can be prepared from acrylic acid crosslinked with allylethers of sucrose or pentaerythritol. The coating of the tablet can have a plurality of formed apertures exposing between about 1 and about 75% of the core surface. Additional substances that can be included in the orally administrable tablets for the controlled release of the active ingredient in a dispersion, as well as methods to make the orally administrable tablets for the controlled release of the active ingredients in a dispersion are described in U.S. Pat. No. 5,882,682.

### Example 10

A tablet for controlled release of the active ingredients through use of a water-soluble alginate salt, and a complex salt of alginic acid and an organic carboxylic acid in admixture with the active ingredients, can be prepared as described in U.S. Pat. No. 5,705,190; wherein the active ingredients as described herein are substituted for the active ingredient described therein. A tablet for a once a day dosage of the active ingredients can be prepared that contains therapeutically effective amounts of the active ingredients, a water-soluble alginate salt, a complex salt of alginic acid, and an organic carboxylic acid. The cation of the alginic acid can be calcium, strontium, iron, or barium. Additional substances that can be included in the orally administrable controlled release tablets, as well as methods to make the orally administrable controlled release tablets are described in U.S. Pat. No. 5,705,190.

### Example 11

An oral composition of the active ingredients can be prepared for targeted slow release of the active ingredients in the intestine, as described in U.S. Pat. No. 5,643,602; wherein the active ingredients as described herein are substituted for the active ingredient described therein. Oral compositions can be prepared that contain the active ingredients in a pellet that contains a core, a layer that surrounds the core, and a membrane that surrounds the layer and the core. The core can contain the active ingredient alone or in combination with other pharmaceutically acceptable materials. The layer surrounding the core can be a pharmaceutically acceptable film-forming, water-insoluble or water-soluble polymer; a pharmaceutically acceptable mixture of film-forming, water-insoluble polymers; or a pharmaceutically acceptable mixture of film-forming, water-soluble and film-forming, water-insoluble polymers. The membrane surrounding both the core and the layer surrounding the core can contain a pharmaceutically acceptable, film-forming, anionic carboxylic polymer that is difficult to dissolve at a low pH but that is soluble at a higher pH of about 4 to 7.5. The polymer of the membrane can be either alone or in combination with a pharmaceutically acceptable, film-forming, water-insoluble polymer. The thickness or the ratio of the anionic carboxylic polymer to the water-insoluble polymer is effective to prevent release of the active ingredient from the pellet in gastric fluids, but permits release of the active ingredient from the pellet in intestinal fluids at a rate allowing treatment of a part of the intestinal tract. Additional substances that can be included in the orally administrable controlled release tablets that can be targeted to the intestine, as well as methods to make the orally administrable controlled release tablets that can be targeted to the intestine are described in U.S. Pat. No. 5,643,602.

### Example 12

A sustained release once-a-day oral formulation of the active ingredients can be prepared that contains therapeutically effective amounts of the active ingredients and a non-aqueous semisolid matrix to impart sustained release properties to the active ingredient, as described in U.S. Pat. No. 5,433,951; wherein the active ingredient as described herein is substituted for the active ingredient described therein. The non-aqueous semisolid matrix is a fatty acid glyceride and/or a polyethylene glycol ester of a fatty acid. The semisolid matrix can be a long chain fatty acid glycerides and/or one or a mixture of polyethylene glycol esters of long chain fatty acids, and mixtures thereof. Additional substances that can be included in the orally administrable sustained release tablets, as well as methods to make the orally administrable sustained release tablets are described in U.S. Pat. No. 5,433,951.

### Example 13

An orally administrable formulation that contains the active ingredients and a permeation-enhancing mixture of sodium salicylate and an oil to provide enhanced absorption of the active ingredients through the wall of the gastrointestinal tract can be prepared as described in U.S. Pat. No. 5,424,289; wherein the active ingredients as described herein are substituted for the active ingredient described therein. The formulation is characterized as a solid, which provides a convenient and improved format for handling and storage and for the preparation of oral dosage forms (such as pills, capsules and delivery vessels) containing a homogeneous mixture of ingredients. The active ingredients can be prepared as a dosage form having an orally administrable, enteric-coated capsule that contains therapeutically effective amounts of the active ingredients, 70-90 weight % of sodium salicylate, and 10-30 weight % of an oil. Additional substances that can be included in the orally administrable tablets, as well as methods to make the orally administrable tablets are described in U.S. Pat. No. 5,424,289.

### Example 14

Oral controlled release dosage units that contain hydroxypropyl methylcellulose can be prepared as described in U.S. Pat. No. 5,419,918; wherein the active ingredients as described herein are substituted for the active ingredient described therein. The aqueous granulation of the dosage units is performed in the presence of one or more solutes, which inhibit gel formation during granulation, but allow formation of a gel when administered orally. Additional substances that can be included in the orally administrable dosage units, as well as methods to make the orally administrable dosage units are described in U.S. Pat. No. 5,419,918.

### Example 15

A mixture of an alginate and a polyacrylate in a ratio of from 15:1 to 1:2 can be prepared as described in U.S. Pat. No. 5,230,901; wherein the active ingredients as described herein are substituted for the active ingredient described therein. Such mixtures are suitable for the preparation of depot drug forms. The active ingredients may be prepared as a tablet for sustained release that includes a blend of a unit dosage of the active ingredients with a mixture of alginate and a polyacrylate in a ratio of 15:1 to 2:1. The polyacrylate can be a copolymer of neutral (meth)acrylic acid esters of methanol, ethanol and trimethylammonioethanol chloride. In addition, the ratio of the ammonium group containing ester unit to the remaining neutral (meth)acrylic acid ester units can be about 1:40. Additional substances that can be included in the tablets, as well as methods to make the tablets, are described in U.S. Pat. No. 5,230,901.

### Example 16

A controlled release pellet containing a core which includes the active ingredients, an intensive disintegrating agent, a wetting agent and a binder; and a double layer which controls release of the activate agents, can be prepared as described in U.S. Pat. No. 5,204,121; wherein the active ingredients as described herein are substituted for the active ingredient described therein. The double layer includes an acrylic-based outer undigestible water-permeable lacquer layer, and an inner jacket layer that contains a hydrophobic additive and hydroxypropylcellulose. The intensive disintegrating agent can be crosslinked sodium carboxymethylcellulose or sodium starch glycolate. The wetting agent can include sodium laurylsulphate. The binder can include PVP. The outer undigestible water-permeable lacquer layer can include an acrylic resin based on a poly(meth)acrylic acid ester having a neutral character or having a low content of quaternary ammonium groups. Such an acid ester can include a copoly(meth)acrylic acid ester, or an ethylcellulose. The inner jacket controls the migration of the water in the direction of the core. The inner jacket can contain hydroxypropylcellulose and a hydrophobic additive that is calcium stearate or hydrogenated castor oil. Additional substances that can be included in the tablets, as well as methods to make the tablets, are described in U.S. Pat. No. 5,204,121.

### Example 17

A sustained release formulation containing the active ingredients and a high and low viscosity HPMC can be prepared as described in U.S. Pat. No. 5,009,895; wherein the active ingredients as described herein are substituted for the active ingredient described therein. The sustained release formulation will exhibit a zero order release profile. A carrier base material can be combined with the active ingredients and shaped and compressed to a solid sustained release pharmaceutical dosage form having a zero order release profile upon administration. The carrier base material can contain a high viscosity hydroxymethylpropylcellulose (HPMC) having a molecular weight of 60,000 or greater; and a low viscosity HPMC, having a molecular weight of 50,000 or less. The high and low viscosity HPMCs are in a ratio yielding a zero order release profile. Additional substances that can be included in the sustained release formulations, as well as methods to make the sustained release formulations, are described in U.S. Pat. No. 5,009,895.

### Example 18

A controlled and sustained release formulation containing a carrier base material combined with the active ingredients can be prepared as described in U.S. Pat. No. 4,983,398; wherein the active ingredients as described herein are substituted for the active ingredient described therein. The carrier base material can contain a mixture of one or more nonionic cellulose ethers and an alkali metal carboxylate. At least one of the cellulose ethers can include hydroxypropylmethylcellulose having a number average molecular weight of at least 50,000. Additional substances that can be included in the sustained release formulations, as well as methods to make the sustained release formulations, are described in U.S. Pat. No. 4,983,398.

### Example 19

A controlled release formulation for the controlled release of the active ingredients can be prepared as described in U.S. Pat. No. 4,946,686; wherein the active ingredients as described herein are substituted for the active ingredient described therein. The formulation includes a core composition containing a plurality of controlled release solubility modulating units that include solubility modulating agents. Each solubility modulating agent is a complexing agent or a surfactant, and is either surrounded by a water insoluble coat containing at least one pore forming additive dispersed throughout, or dispersed in an individual matrix substrate. Each unit also includes the active ingredients, and a water insoluble microporous wall that surrounds the core composition. The water insoluble microporous wall contains a polymer material that is permeable to water but substantially impermeable to solute, and at least one water leachable pore forming additive dispersed throughout the wall. Additional substances that can be included in the controlled release formulations, as well as methods to make the controlled release formulations, are described in U.S. Pat. No. 4,946,686.

### Example 20

An oral sustained release tablet having a core and a coating layer can be prepared as described in described in U.S. Pat. No. 4,919,938; wherein the active ingredients as described herein are substituted for the active ingredient described therein. The core matrix can contain 20% to 60% w/w of a hydroxypropylmethylcellulose gelling agent, 0.41% to 20% w/w of (+)-trans-1a,2,3,4a,5,6-hexahydro-9-hydroxy-4-(1-propyl)-4H-naphth[1,2-b]-1,4-oxazine hydrochloride, and 2.08 to 12.5% w/w of buffering agent homogeneously dispersed therein. The core can also include suitable pharmaceutically acceptable excipients. The coating layer surrounding the core matrix can include a slowly soluble, water permeable ethyl cellulose polymer. Additional substances that can be included in the controlled release tablets, as well as methods to make the controlled release tablets, are described in U.S. Pat. No. 4,919,938.

### Example 21

A solid unit dosage form having a controlled and prolonged release pattern upon administration can be prepared as described in U.S. Pat. No. 4,849,229; wherein the active ingredients as described herein are substituted for the active ingredient described therein. The dosage form can contain a mixture of a high viscosity grade methylcellulose or hydroxypropylmethylcellulose, an alkali metal sulfate or sulfonate and the active ingredients. A therapeutically active solid unit dosage form having a controlled and prolonged release pattern upon administration, can contain a mixture of a high viscosity grade water-soluble nonionic cellulose ether having a number average molecular weight of at least 50,000 and a methoxyl content of 16.5-31.5 weight-%. The cellulose ether can include methylcellulose, hydroxypropylmethylcellulose, or mixtures thereof. The dosage form can also include an alkali metal sulfonate of aliphatic and aromatic hydrocarbons and succinic esters, and the active ingredient. Additional substances that can be included in the dosage form, as well as methods to make the dosage form, are described in U.S. Pat. No. 4,849,229.

### Example 22

A controlled, slow release, solid pharmaceutical composition that includes the active ingredients and a blend of sodium alginate and sodium-calcium alginate can be prepared as described in U.S. Pat. No. 4,842,866; wherein the active ingredients as described herein are substituted for the active ingredient described therein. Additional substances that can be included in the dosage form, as well as methods to make the dosage form, are described in U.S. Pat. No. 4,842,866.

### Example 23

A controlled and prolonged release composition having a carrier base material that is combined with the active ingredients and shaped and compressed to a solid unit dosage form can be prepared as described in U.S. Pat. No. 4,795,327; wherein the active ingredients as described herein are substituted for the active ingredient described therein. The carrier base material is a mixture of one or more nonionic cellulose ethers and an anionic surfactant. At least one of the cellulose ethers is methyl cellulose or hydroxypropylmethylcellulose having a number average molecular weight of at least 50,000 and a methoxyl content of 16.5-31.5 weight-%. Additional substances that can be included in the dosage form, as well as methods to make the dosage form, are described in U.S. Pat. No. 4,795,327.

### Example 24

A hydrogel reservoir containing pills that provide for controlled delivery of the active ingredients can be prepared as described in U.S. Pat. No. 4,649,043; wherein the active ingredients as described herein are substituted for the active ingredient described therein. The pills include a wall surrounding a core of the active ingredients. The hydrogel reservoir includes a matrix that contains a pharmaceutically acceptable non-toxic, non-hydrated polyethylene oxide that exhibits the ability to retain fluid within its polyethylene oxide structure, absorb fluid from the gastrointestinal tract, and expand with at least a 2 fold volume increase for retaining the hydrogel reservoir in the stomach over an extended period of time. The hydrogel reservoir includes a plurality of pills dispensed throughout the matrix of the reservoir. The pills contain a dosage amount of the active ingredients and a wall containing a release rate controlling composition that contains a cellulosic polymer that surrounds the dosage amount of the active ingredients. The matrix can contain a pharmaceutically acceptable non-toxic, non-hydrated carboxy polymer that exhibits the ability to retain fluid within its carboxy polymer structure, absorb fluid from the gastrointestinal tract, and expand with at least a 2 fold volume increase for retaining the dispensing device in the stomach over an extended period of time. Additional substances that can be included in the hydrogel reservoirs, as well as methods to make the hydrogel reservoirs, are described in U.S. Pat. No. 4,649,043.

### Example 25

A sustained release composition that is made from a plurality of pellets can be prepared as described in U.S. Pat. No. 4,634,587; wherein the active ingredients as described herein are substituted for the active ingredient described therein. Each pellet can include the active ingredient-containing coating over a nonpareil seed, with a further coating of about 5 to about 15% by weight of a mixture of about 1.5 to about 9 parts by weight ethylcellulose to about 1 part by weight hydroxypropylcellulose. Additional substances that can be included in the sustained release compositions, as well as methods to make the sustained release compositions, are described in U.S. Pat. No. 4,634,587.

### Example 26

A sustained release oral formulation that contains a capsule that includes upper and lower parts that are connectible and easily separable from each other, and a plurality of micropellets present in the capsule, can be prepared as described in U.S. Pat. No. 4,587,118; wherein the active ingredients as described herein are substituted for the active ingredient described therein. The micropellets provide sustained release of the active ingredient when taken by a patient. The micropellets contain inner seeds coated with a mixture of theophylline and polyvinylpyrrolidone which is further coated with a mixture of ethylcellulose and hydroxypropylcellulose. Additional substances that can be included in the sustained release compositions, as well as methods to make the sustained release compositions, are described in U.S. Pat. No. 4,587,118.

### Example 27

A sustained release tablet for oral administration can be prepared as described in U.S. Pat. No. 4,556,678; wherein the active ingredients as described herein are substituted for the active ingredient described therein. The tablet contains compressed granules that include the active ingredients, from about 0.1 to about 10 parts by weight hydroxypropyl methylcellulose, about one part by weight hydroxypropyl cellulose, and a lubricant. The hydroxypropyl methylcellulose will have a molecular weight of from about 20,000 to about 140,000. The hydroxypropyl cellulose will have a molecular weight of from about 60,000 to about 300,000. Additional substances that can be included in the sustained release compositions, as well as methods to make the sustained release compositions, are described in U.S. Pat. No. 4,556,678.

### Example 28

An oral unit dosage containing a carrier base material and the active ingredients for controlled and prolonged release can be prepared as described in U.S. Pat. No. 4,540,566; wherein the active ingredients as described herein are substituted for the active ingredient described therein. The carrier base material can be a mixture of one or more nonionic cellulose ethers and an anionic surfactant. At least one of the cellulose ethers can be a modified hydroxypropylmethylcellulose having a number average molecular weight of less than 50,000 and has been modified by successive or concurrent exposure to moisture and air. Additional substances that can be included in the sustained release compositions, as well as methods to make the sustained release compositions, are described in U.S. Pat. No. 4,540,566.

### Example 29

A sustained release composition that contains a plurality of polymerically coated seeds of the active ingredient can be prepared as described in U.S. Pat. No. 4,508,702; wherein the active ingredients as described herein are substituted for the active ingredient described therein. Each of the seeds can be individually coated with a polymeric mixture, which contains from about 1.5 to about 15 parts by weight ethylcellulose and about one part by weight hydroxypropylcellulose. Additional substances that can be included in the sustained release compositions, as well as methods to make the sustained release compositions, are described in U.S. Pat. No. 4,508,702.

### Example 30

A self-supporting polymeric diffusion matrix that provides for the sustained release of the active ingredient can be prepared as described in U.S. Pat. No. 4,482,533; wherein the active ingredients as described herein are substituted for the active ingredient described therein. The matrix can contain from about 1 to about 60% by weight of a polar plasticizer; from about 5 to about 20% by weight polyvinylalcohol having a molecular weight from about 50,000 to about 150,000; from about 10 to about 25% by weight polyvinylalcohol having a molecular weight from about 4,000 to about 15,000; from about 2 to about 30% by weight polyvinylpyrrolidone; a pharmaceutically effective amount of the active ingredient to provide a sustained release of the active ingredient over a prolonged period; and from about 5 to about 20% by weight of diethanol myristoylamide. The diethanol myristoylamide can function to bring the components into solution. Additional substances that can be included in the sustained release matrixes, as well as methods to make the sustained release matrixes, are described in U.S. Pat. No. 4,482,533.

### Example 31

A sustained release oral dosage form as a tablet having a core that contains pharmaceutically effective amounts of the active ingredients can be prepared as described in U.S. Pat. No. 4,432,965; wherein the active ingredients as described herein are substituted for the active ingredient described therein. The tablet core can be coated with a sustained release polymeric coating which contains about 5 to about 20 percent by weight polyethylene glycol component having a molecular weight of from about 500 to about 2000, and from about 80 to 95 percent by weight polyvinylalcohol component. The polyvinylalcohol component can contain from about one to about ten parts by weight of a partially hydrolyzed polyvinylalcohol subcomponent having a molecular weight of from about 50,000 to about 110,000 and having a degree of hydrolysis of from about 75 to about 92 percent. The polyvinylalcohol component can also contain about one part by weight of a substantially completely hydrolyzed polyvinylalcohol subcomponent having a molecular weight of from about 90,000 to about 150,000 and having a degree of hydrolysis in excess of 95%. Additional substances that can be included in the sustained release oral dosage forms, as well as methods to make the sustained-release oral dosage forms, are described in U.S. Pat. No. 4,432,965.

### Example 32

Pharmaceutical tablets, lozenges, suppositories and other solid dosage unit forms that have a prolonged and regular release pattern of the active ingredient can be prepared as described in U.S. Pat. No. 4,226,849; wherein the active ingredients as described herein are substituted for the active ingredient described therein. The shaped dosage unit can contain a carrier base material of hydroxypropylmethylcellulose or a mixture thereof with up to 30% ethylcellulose and/or up to 30% sodium carboxymethylcellulose. The carrier base material can be subjected to hydrolysis and oxidation, so as to generate a desired minimum concentration of carbonyl and carboxyl groups, and then admixed and shaped with the active ingredient of the invention. Additional substances that can be included in the sustained release dosage forms, as well as methods to make the sustained release dosage forms, are described in U.S. Pat. No. 4,226,849.

### Example 33

A sustained release composition that utilizes a pellet formulation encapsulated in a hard gelatin capsule can be prepared as described in U.S. Pat. No. 4,173,626; wherein the active ingredients as described herein are substituted for the active ingredient described therein. A portion of the pellets can be uncoated for immediate and rapid release of the active ingredient for elevating the plasma level of the active ingredient. The remainder of the pellets can be coated with a polymer to sustain the plasma levels of the active ingredients. The uncoated and coated pellets may be mixed with non-medicated pellets as a capsule filler. Additional substances that can be included in the sustained release compositions, as well as methods to make the sustained release compositions, are described in U.S. Pat. No. 4,173,626.

### Example 34

A controlled release solid dosage composition can be prepared as described in U.S. Pat. No. 6,365,196; wherein the active ingredients as described herein are substituted for the active ingredient described therein. The composition can include a dissolution rate stabilizer and a hydrophobic waxy material. The composition can contain about 40 to 90% by weight of the active ingredients, a hydrophobic waxy material in about 5 to 30% by weight, a dissolution rate stabilizer in an amount greater than 1% to about 15% by weight; and optional pharmaceutically acceptable excipients. Additional substances that can be included in the above compositions, as well as methods to make the compositions are described in U.S. Pat. No. 6,365,196.

### Example 35

A stabilized solid controlled release dosage form can be prepared as described in U.S. Pat. No. 6,316,031; wherein the active ingredients as described herein are substituted for the active ingredient described therein. The controlled release dosage form can have an inert bead coated with the active ingredients, a barrier layer over the bead that is coated with the active ingredients, and a controlled release layer that is added over the barrier layer. The barrier layer can include hydroxypropylmethylcellulose. The barrier layer can be coated with a controlled release layer derived from an aqueous dispersion of plasticized ethylcellulose in an amount sufficient to obtain controlled release of the active ingredient when the bead is exposed to a gastrointestinal fluid. The coated bead will be cured at a temperature greater than the glass transition temperature of the plasticized ethylcellulose for at least about 24 hours. This will cause individual ethylcellulose particles in the coating to coalesce and to gradually slow the release of the active ingredients when the bead is exposed to aqueous fluid until an endpoint is reached. When the endpoint is reached, the active ingredients will be released in amounts which do not significantly vary at any time point along the dissolution curve by more than about 20% of the total amount of the active ingredients released, when compared to the in-vitro dissolution of the coated bead prior to curing. Additional substances that can be included in the above compositions, as well as methods to make the compositions are described in U.S. Pat. No. 6,316,031.

### Example 36

A stable solid controlled release composition can be prepared as described in U.S. Pat. No. 6,143,353; wherein the active ingredients as described herein are substituted for the active ingredient described therein. The stable solid controlled release composition will have a coating derived from an aqueous dispersion of a hydrophobic acrylic polymer that includes a substrate containing the active ingredients that is overcoated with an aqueous dispersion of a plasticized water-insoluble acrylic polymer. The composition will provide stable dissolution of the active ingredients that is unchanged after exposure to accelerated storage conditions. The plasticized water-insoluble acrylic polymer contains monomers that can be, for example, an ester of acrylic acid, an ester of methacrylic acid, an alkyl ester of acrylic acid, an alkyl ester of methacrylic acid, and mixtures of any of the foregoing. The compositions can include an additional material that is a polymerizable permeability-enhancing agent, a water-soluble acrylic polymer, a pore-former, and mixtures of any of the foregoing. This will provide controlled release of the active ingredients when the coated substrate is exposed to an environmental fluid. Additional substances that can be included in the above compositions, as well as methods to make the compositions are described in U.S. Pat. No. 6,143,353.

### Example 37

A controlled release composition having microparticles that contain the active ingredients in a polymeric matrix can be prepared as described in U.S. Pat. No. 5,688,530; wherein the active ingredients as described herein are substituted for the active ingredient described therein. The polymeric matrix is a biodegradable, biocompatible polymeric matrix of a 40/60 to 60/40 polylactide-co-glycolide ester of a polyol. The polyol is a (C.sub.3-6) carbon chain containing alcohol having 3 to 6 hydroxyl groups, or a mono-saccharide and a disaccharide. The esterified polyol will have at least 3 polylactide-co-glycolide chains. Additional substances that can be included in the above compositions, as well as methods to make the compositions are described in U.S. Pat. No. 5,688,530.

### Example 38

A capsule containing a plurality of coated particles that contain therapeutically effective amounts of the active ingredients can be prepared as described in U.S. Pat. No. 5,656,291; wherein the active ingredients as described herein are substituted for the active ingredient described therein. The particles are coated with a barrier membrane providing a controlled, preferably pH-independent, release of the active ingredients. The particles will contain at least one water insoluble component (e.g. ethyl cellulose, copolymers of acrylic and methacrylic esters, or natural or synthetic waxes). The water insoluble component will provide a pH-independent drug release. Additional substances that can be included in the above compositions, as well as methods to make the compositions are described in U.S. Pat. No. 5,656,291.

### Example 39

A multilayered controlled release pharmaceutical dosage composition can be prepared as described in U.S. Pat. No. 5,645,858; wherein the active ingredients as described herein are substituted for the active ingredient described therein. The multilayered controlled release pharmaceutical dosage composition contains a plurality of coated particles. Each particle contains a core that will contain the active ingredients and a mixture of hydroxypropyl methylcellulose, polyethylene glycol and propylene glycol. The core will be overcoated with a controlled release barrier layer that will contain ethyl cellulose. The controlled release barrier that coats the core will be overcoated with another layer that contains the active ingredients and a mixture of hydroxypropyl methylcellulose, polyethylene glycol and propylene glycol. The second layer that contains the active ingredients will be overcoated with another controlled release barrier layer that will contain ethyl cellulose. Additional substances that can be included in the above compositions, as well as methods to make the compositions are described in U.S. Pat. No. 5,645,858.

### Example 40

A sustained release homogeneous tablet or homogeneous tablet layer can be prepared as described in U.S. Pat. No. 5,462,747; wherein the active ingredients as described herein are substituted for the active ingredient described therein. The table or tablet layer can be formed by making a wet granulation using povidone (PVP) in alcohol as the granulating fluid. The wet granulation can then be dried, milled, and blended with a dry powdered erosion promoter, wicking agent, lubricant, and a glidant. The mixture can be compressed to produce a tablet or tablet coating which, upon administration to a patient, results in a long-lasting slow and relatively regular incremental release of the active ingredient. The mixture can be used to produce multilayer tablets for immediate release and sustained release of the active ingredient. An example of a wicking agent is microcrystalline cellulose. An example of an erosion promoter is pregelatinized starch. An example of a lubricant is magnesium stearate. An example of a glidant is silicon dioxide. Additional substances that can be included in the above compositions, as well as methods to make the compositions are described in U.S. Pat. No. 5,462,747.

### Example 41

A sustained release homogeneous tablet or homogeneous tablet layer can be prepared as described in U.S. Pat. No. 5,393,765; wherein the active ingredients as described herein are substituted for the active ingredient described therein. The active ingredients of the invention can be prepared as an erodible pharmaceutical composition providing a unique zero order controlled release profile. The erodible composition can contain between about 5% to about 60% w/w of the active ingredients which have a solubility of less than about 80 mg/mL. The composition can also contain about 5% to about 50% w/w of hydroxypropyl methylcellulose having a viscosity from about 50 to about 100 centipoises. The remainder of the composition will consist of inert carriers. Additional substances that can be included in the above compositions, as well as methods to make the compositions are described in U.S. Pat. No. 5,393,765.

### Example 42

A composition for the sustained release of the active ingredients can be prepared as described in U.S. Pat. No. 5,356,635; wherein the active ingredients as described herein are substituted for the active ingredient described therein. The composition includes an amorphous carbohydrate glass matrix containing a suitable carbohydrate and the active ingredients which retard the recrystallization of the carbohydrate and the active ingredients. The matrix will also have a water-insoluble wax dispersed throughout the matrix. Additional substances that can be included in the above compositions, as well as methods to make the compositions are described in U.S. Pat. No. 5,356,635.

### Example 43

A composition for the sustained release of the active ingredients can be prepared as described in U.S. Pat. No. 5,328,697; wherein the active ingredients as described herein are substituted for the active ingredient described therein. The composition will have the active ingredient layered onto non-pareil seeds which are sprayed with a glycine solution. Next, a coating of a wax mixture is applied. Additional substances that can be included in the above compositions, as well as methods to make the compositions are described in U.S. Pat. No. 5,328,697.

### Example 44

A stable sustained release the active ingredient-resin composition for use in liquid carrier for oral administration can be prepared as described in U.S. Pat. No. 5,186,930; wherein the active ingredients as described herein are substituted for the active ingredient described therein. The composition contains the active ingredients-resin particle that is coated with a first inner coating of a high temperature melting water-insoluble pharmaceutically acceptable wax and a second outer coating of a pharmaceutically acceptable water-insoluble polymer. The active ingredients-resin particle contains the active ingredients tonically bonded to a pharmaceutically acceptable ion exchange resin particle. The amount of the first inner coating is sufficient to prevent the resin in the active ingredients-resin particle from swelling and cracking the second outer coating. The active ingredients are released when the complex is placed in a liquid carrier. Additional substances that can be included in the above compositions, as well as methods to make the compositions are described in U.S. Pat. No. 5,186,930.

### Example 45

A stable sustained release the active ingredients-resin composition for use in a liquid carrier for oral administration can be prepared as described in U.S. Pat. No. 4,892,742; wherein the active ingredients as described herein are substituted for the active ingredient described therein. The controlled release composition in table form contains a core element that includes about 65-95% by weight of water soluble active ingredients, 5-35% by weight of a water insoluble polymeric matrix; and a membrane coating comprising 5-10% by weight of the tablet. The membrane contains a rate-controlling polymer. The insoluble polymeric matrix can contain ethyl cellulose or zein. The insoluble polymer matrix can also contain an oil or wax-like material (e.g. stearic acid, stearyl alcohol, cetyl alcohol, fatty acids, long chain fatty alcohols, carnuba wax, beeswax, white wax, vegetable oil and fatty acid glycerides of C.sub.6-18 fatty acids). The membrane coating can be cellulose (e.g. ethyl cellulose, mixtures of ethyl cellulose and hydroxypropyl methylcellulose or hydroxypropyl cellulose). The membrane coating can further contain a plasticizer (e.g. triacetin, propylene glycol, polyethylene glycol having a molecular weight of 200 to 800, dibutyl phthalate, dibutyl sebacate, fatty acid, vegetable oils and glycerides of C.sub.6-18 fatty acids). Additional substances that can be included in the above compositions, as well as methods to make the compositions are described in U.S. Pat. No. 4,892,742.

### Example 46

A stable sustained release dosage composition for use in a liquid carrier for oral administration can be prepared as described in U.S. Pat. No. 4,781,919; wherein the active ingredients as described herein are substituted for the active ingredient described therein. The dosage compositions are made of saponified starch-acrylonitrile graft copolymers and the active ingredients. The sustained release injectable dosage forms can contain therapeutically effective amounts of the active ingredients, and a therapeutically effective amount of a water insoluble, water swellable, saponified starch acrylonitrile graft copolymer to provide sustained release of the active ingredients upon injection into a patient in need of such treatment. Additional substances that can be included in the above compositions, as well as methods to make the compositions are described in U.S. Pat. No. 4,781,919.

### Example 47

A controlled release dosage composition containing the active ingredients in combination with hydroxypropylmethylcellulose USP 2910 can be prepared as described in U.S. Pat. No. 4,695,591; wherein the active ingredients as described herein are substituted for the active ingredient described therein. The hydroxypropylmethylcellulose USP 2910 can be less than about one-third of the total dosage form weight of hydroxypropylmethylcellulose USP 2910. Additional substances that can be included in the above compositions, as well as methods to make the compositions are described in U.S. Pat. No. 4,695,591.

### Example 48

A controlled release dosage composition containing a plurality of nicronized pellets can be prepared as described in U.S. Pat. No. 4,524,060; wherein the active ingredients as described herein are substituted for the active ingredient described therein. The micronized pellets will contain the active ingredients, a water-channeling agent, a wetting agent, and a disintegrant. The mixture can be in the form of a non-compressed pellet having an enteric coat or a sustained release coat permeable to gastrointestinal juices. The micronized pellets can be placed into sustained-release capsules. Additional substances that can be included in the above compositions, as well as methods to make the compositions are described in U.S. Pat. No. 4,524,060.

### Example 49

A controlled release pharmaceutical composition can be prepared as described in U.S. Pat. No. 6,491,950; wherein the active ingredients as described herein are substituted for the active ingredient described therein. The composition can include a matrix of a material that includes a high melting point fatty acid ester, an oil, a polymeric cellulose derivative, or a combination thereof. The active ingredients can optionally be associated with the matrix. The formulation can optionally include a surfactant (e.g., polysorbate 80). Suitable high melting fatty acid esters include, e.g., glyceryl behenate, glyceryl palmitostearate, and glyceryl stearate. Suitable oils include, e.g., corn oil, cottonseed oil, menhaden oil, safflower oil, sesame oil, shark-liver oil, soybean oil, olive oil, and wheat germ oil. Suitable cellulosic polymers include, e.g., a low-substituted hydroxypropyl ether cellulose polymer and a cellulosic polymer having methylether substitution. Suitable high melting fatty acid esters include, e.g., glyceryl behenate, glyceryl palmitostearate and glyceryl stearate. Additional substances that can be included in the above pharmaceutical compositions, as well as methods to make the pharmaceutical compositions, are described in U.S. Pat. No. 6,491,950.

### Example 50

A biphasic controlled release pharmaceutical composition can be as described in U.S. Pat. No. 6,475,521; wherein the active ingredients as described herein are substituted for the active ingredient described therein. Such a system can provide a dosage form that has prolonged gastric residence so that the active ingredients can be administered once daily to sustain a continuous plasma concentration of the active ingredient. The controlled release pharmaceutical composition includes an inner solid particulate phase formed of substantially uniform granules containing the active ingredients, one or more hydrophilic polymers, and one or more hydrophobic polymers. The delivery system can also include one or more hydrophobic materials, such as one or more waxes, fatty alcohols and/or fatty acid esters. The controlled release pharmaceutical composition has an outer solid continuous phase in which the above granules of inner solid particulate phase are embedded and dispersed throughout. This outer solid continuous phase includes one or more hydrophilic polymers, one or more hydrophobic polymers and/or one or more hydrophobic materials such as one or more waxes, fatty alcohols and/or fatty acid esters. The controlled release pharmaceutical composition may be compressed into tablets or filled into capsules. The particles of the inner solid particulate phase can include the active ingredient and an extended release material. The outer solid continuous phase can include an extended release material. Additional substances that can be included in the above pharmaceutical compositions, as well as methods to make the pharmaceutical compositions, are described in U.S. Pat. No. 6,475,521.

### Example 51

A controlled release tablet form, having a hydrophilic matrix that is suitable for the once-a-day administration, can be prepared as described in U.S. Pat. No. 6,419,953; wherein the active ingredients of the present invention are substituted for the active ingredient described therein. The tablet can include from about 50 weight percent to about 55 weight percent of the active ingredient, from about 20 weight percent to about 40 weight percent hydroxypropyl methylcellulose, from about 5 weight percent to about 15 weight percent lactose, from about 4 weight percent to about 6 weight percent microcrystalline cellulose, and from about 1 weight percent to about 5 weight percent of silicon dioxide. All of the weight percentages are based upon the total weight of the tablet dosage form. More specifically, the controlled release tablet can be formed from a uniform admixture of about 54 weight percent of the active ingredients, about 30 weight percent hydroxypropyl methylcellulose, about 8 weight percent lactose, about 5 weight percent microcrystalline cellulose, and about 3 weight percent silicon dioxide. More specifically, the controlled release tablet can also be formed from a uniform admixture of about 54 weight percent of the active ingredients, about 30 weight percent hydroxypropyl methylcellulose, about 8 percent lactose, about 5 weight percent microcrystalline cellulose, and about 3 weight percent silicon dioxide. Additional substances that can be included in the above controlled release tablets, as well as methods to make the controlled release tablets, are described in U.S. Pat. No. 6,419,953.

### Example 52

A controlled release gelatin capsule formed with a composite wall that contains a liquid, the active ingredients formulation can be prepared as described in U.S. Pat. No. 6,419,952; wherein the active ingredients as described herein are substituted for the active ingredient described therein. The composite wall includes a barrier layer formed over the external surface of the gelatin capsule, an expandable layer formed over the barrier layer, and a semipermeable layer formed over the expandable layer. The controlled release gelatin capsule includes a gelatin capsule containing a liquid, the active ingredients formulation; and a multilayer wall superposed on the gelatin capsule. The multilayer wall includes a deformable barrier layer, an expandable layer, a semipermeable layer; and an orifice formed or formable through the wall. Additional substances that can be included in the above controlled release gelatin capsules, as well as methods to make the controlled release gelatin capsules, are described in U.S. Pat. No. 6,419,952.

Additional formulations that can be prepared to include the active ingredients, and methods of preparing the formulations are described, e.g., in U.S. Pat. Nos. 6,419,953; 6,251,432; 6,197,344; 6,150,410; 6,033,685; 6,010,718; 5,705,190; 5,268,182; 5,169,642; 6,419,952; 6,395,292; 6,375,978; 6,368,626; 6,342,249; 6,245,357; 6,174,547; 6,077,538; 5,650,170; 5,540,912; 5,512,293; 4,871,548; 4,740,198; 4,692,144; 6,270,799; 5,900,425; 5,707,655; 5,204,121; 5,368,862; 5,366,738; 5,009,895; 4,983,400; 4,919,938; 4,900,755; 4,832,957; 4,639,458; 4,173,626; 5,690,960; 5,660,837; 5,419,918; 4,863,743; 4,634,587; 4,587,118; 4,556,678; 4,508,702; 4,432,965; 4,428,926; 4,428,925; 6,500,454; 6,495,162; 6,492,488; 6,437,000; 6,426,091; 6,419,958; 6,419,953; 6,419,952; 6,416,786; 6,403,120; 6,387,404; 6,372,252; 6,337,091; 6,303,144; 6,284,275; 6,274,171; 6,261,601; 6,254,891; 6,221,395; 6,210,714; 6,197,339; 6,162,466; 6,162,463; 6,156,343; 6,150,410; 6,149,940; 6,136,343; 6,126,967; 6,106,863; 6,099,862; 6,099,859; 6,093,387; 6,090,411; 6,083,533; 6,074,669; 6,056,977; 6,046,177; 6,033,686; 6,033,685; 6,030,642; 6,030,641; 6,027,748; 6,024,982; 5,980,942; 5,945,125; 5,885,615; 5,879,707; 5,874,107; 5,869,100; 5,849,330; 5,846,563; 5,783,212; 5,776,489; 5,736,159; 5,681,583; 5,681,582; 5,667,801; 5,656,291; 5,654,005; 5,645,848; 5,626,874; 5,624,683; 5,614,218; 5,603,956; 5,601,842; 5,593,694; 5,582,837; 5,578,321; 5,576,021; 5,562,915; 5,558,879; 5,554,387; 5,543,155; 5,512,297; 5,508,041; 5,505,962; 5,500,227; 5,498,422; 5,492,700; 5,484,607; 5,466,460; 5,462,747; 5,455,046; 5,433,951; 5,427,799; 5,427,798; 5,407,686; 5,397,574; 5,368,862; 5,362,424; 5,358,723; 5,334,393; 5,334,392; 5,292,534; 5,292,533; 5,283,065; 5,277,912; 5,219,572; 5,200,193; 5,164,193; 5,162,117; 5,126,145; 5,091,189; 5,085,865; 5,075,114; 5,073,380; 5,055,306; 5,051,261; 5,019,398; 5,015,479; 5,007,790; 5,004,613; 5,002,774; 4,983,401; 4,968,509; 4,966,768; 4,933,185; 4,925,676; 4,892,742; 4,882,167; 4,861,590; 4,837,032; 4,824,678; 4,822,619; 4,820,522; 4,816,262; 4,806,359; 4,803,079; 4,803,076; 4,800,083; 4,798,725; 4,795,645; 4,795,642; 4,792,448; 4,784,858; 4,775,535; 4,756,911; 4,734,285; 4,710,384; 4,708,834; 4,695,467; 4,692,337; 4,690,824; 4,666,705; 4,629,620; 4,629,619; 4,610,870; 4,587,118; 4,571,333; 4,557,925; 4,556,678; 4,520,009; 4,505,890; 4,503,031; 4,432,965; 4,415,547; 4,353,887; 4,322,311; 4,308,251; 4,264,573; 4,252,786; 4,173,626; 4,138,475; 4,122,157; 4,002,458; and 3,977,992.

Alternatively, the active ingredient can be administered in a formulation that will form a biodegradable or bioerodible implant, either ex vivo or in vivo. The biodegradable or bioerodible implant, upon degrading in vivo, will release the active ingredient over a suitable period of time. Such formulations that will form a biodegradable implant, either ex vivo or in vivo, are described, e.g., in U.S. Pat. Nos. RE37,950; 6,461,631; 6,395,293; 6,261,583; 6,180,129; 6,143,314; 6,120,789; 6,113,938; 6,071,530; 5,990,194; 5,945,115; 5,888,533; 5,861,166; 5,780,044; 5,759,563; 5,744,153; 5,739,176; 5,736,152; 5,733,950; 5,702,716; RE35,601; 5,630,808; 5,599,552; 5,487,897; 5,413,572; 5,368,859; 5,340,849; 5,324,519; 5,320,616; 5,278,202; 5,278,201; 5,238,687; 5,234,693; 5,234,692; 5,137,727; 5,112,614; 5,057,318; 4,996,060; 4,455,144; 4,367,741; 4,346,709; 4,340,054; 4,304,232; 4,249,531; 4,142,526; 4,093,709; 4,069,307; and 3,948,254.

Any patent, patent document, or reference disclosed herein is incorporated into reference into this invention and forms part of the invention. Obviously, numerous modifications and variations of the present invention are possible in light of the above teachings. It is therefore to be understood that within the scope of the appended claims, the invention may be practiced otherwise than as specifically described herein.

What is claimed is:

1. A composition comprising:
(a) a therapeutically effective amount of at least one alpha2-adrenergic agonist or a pharmaceutically acceptable derivative thereof; and
(b) a therapeutically effective amount of at least one alpha 1-adrenergic agonist or a pharmaceutically acceptable derivative thereof.

2. The composition of claim 1, wherein the alpha2-adrenergic agonist is present in an amount sufficient to at least partially impart muscle relaxation in a human patient.

3. The composition of claim 1, wherein the alpha2-adrenergic agonist is present in an amount sufficient to at least partially alleviate pain in a human patient.

4. The composition of claim 1, wherein the alpha1-adrenergic agonist is present in an amount sufficient to at least partially impart stimulation of the nervous system in a human patient.

5. A composition comprising:
(a) a therapeutically effective amount of tizanidine or a pharmaceutically acceptable derivative thereof;
(b) a therapeutically effective amount of modafinil or a pharmaceutically acceptable derivative thereof; and
(c) a pharmaceutically acceptable excipient.

6. The composition of claim 5, wherein the composition is in a form of tablet or capsule.

7. The composition of claim 6, wherein the tablet comprises at least one coating of tizanidine or a pharmaceutically acceptable derivative thereof and at least one coating of modafinil or a pharmaceutically acceptable derivative thereof.

8. The composition of claim 6, wherein the capsule comprises particles comprising tizanidine or a pharmaceutically acceptable derivative thereof and/or modafinil or a pharmaceutically acceptable derivative thereof.

9. A composition comprising:
(a) a therapeutically effective amount of baclofen or a pharmaceutically acceptable derivative thereof; and
(b) a therapeutically effective amount of at least one alpha1-adrenergic agonist or a pharmaceutically acceptable derivative thereof.

10. The composition of claim 9, wherein baclofen is present in an amount sufficient to at least partially impart muscle relaxation in a human patient.

11. The composition of claim 9, wherein baclofen is present in an amount sufficient to at least partially alleviate pain in a human patient.

12. The composition of claim 9, wherein the alpha1-adrenergic agonist is present in an amount sufficient to at least partially impart stimulation of the central nervous system in a human patient.

13. The composition of claim 9, wherein the alpha1-adrenergic agonist is modafinil.

14. A method of reducing somnolence in a patient receiving an alpha2-adrenergic agonist therapy comprising administering to the patient a composition comprising:
(a) a therapeutically effective amount of at least one alpha1-adrenergic agonist or a pharmaceutically acceptable derivative thereof; and
(b) a pharmaceutically acceptable excipient.

15. The method of claim 14, wherein the alpha1-adrenergic agonist is present in an amount sufficient to at least partially impart stimulation of the central nervous system in a human patient.

16. The method of claim 14, wherein the alpha1-adrenergic agonist is modafinil.

17. The method of claim 14, wherein the alpha2-adrenergic agonist is tizanidine.

18. A method for treating pain comprising administering to a patient in need thereof:
(a) a therapeutically effective amount of at least one alpha2-adrenergic agonist or a pharmaceutically acceptable derivative thereof; and
(b) a therapeutically effective amount of at least one alpha1-adrenergic agonist or a pharmaceutically acceptable derivative thereof.

19. The method of claim 18, wherein the alpha2-adrenergic agonist is tizanidine and the alpha1-adrenergic agonist is modafinil.

20. A method for attenuating muscle spasticity comprising administering to a patient in need thereof:
(a) a therapeutically effective amount of at least one alpha2-adrenergic agonist or a pharmaceutically acceptable derivative thereof; and
(b) a therapeutically effective amount of at least one alpha1-adrenergic agonist or a pharmaceutically acceptable derivative thereof.

21. The method of claim 20, wherein the alpha2-adrenergic agonist is tizanidine and the alpha1-adrenergic agonist is modafinil.
